(19) United States
(12) Patent Application Publication
(10) Pub. No.: US 2023/0043204 A1
(43) Pub. Date: Feb. 9, 2023

(54) DIHYDROERGOTAMINE MESYLATE FORMULATIONS AND PRE-FILLED INJECTORS FOR THERAPEUTIC DELIVERY OF THE SAME

(71) Applicant: Scienture, Inc., Hauppauge, NY (US)

(72) Inventors: Shankar HARIHARAN, Smithtown, NY (US); Bhavya Teja KOLLA, South Setauket, NY (US); Suketu SANGHVI, Kendall Park, NJ (US); Rahul SURANA, Commack, NY (US)

(21) Appl. No.: 17/757,924

(22) PCT Filed: Dec. 21, 2020
(86) PCT No.: PCT/US2020/066482

§ 371 Date: Jun. 23, 2022

Related U.S. Application Data
(60) Provisional application No. 62/952,925, filed on Dec. 23, 2019.

(51) Int. Cl. A61K 31/48; A61K 47/26; A61K 47/18; A61K 47/10; A61P 25/06; A61K 9/00; A61M 5/178; A61K 31/522

(52) U.S. Cl. CPC A61K 31/48; A61K 47/26; A61K 47/183; A61K 47/10; A61P 25/06; A61K 9/0019; A61M 5/178; A61K 31/522

(57) ABSTRACT

The present disclosure is directed to formulations and methods for treating or preventing head pain, including migraines, with dihydroergotamine mesylate.

### FIELD OF THE INVENTION

According to various aspects of this disclosure, the present disclosure relates to pre-filled injectors comprising pharmaceutically acceptable stable formulations comprising dihydroergotamine mesylate, methods of producing the formulations, and methods of treating head pain with the pharmaceutically acceptable formulations.

### BACKGROUND OF THE INVENTION

Ergotamine is an ergot alkaloid first isolated in 1918 from the fungus Claviceps purpurea. Ergotamine was reported as an effective antimigraine drug in 1926. However, because of its unfavorable side-effect profile, the dihydrogenated derivative dihydroergotamine (DHE) was developed for treatment of migraine (Horton, B. T., Peters, G. A., and Blumenthal, L. S. 1945. Mayo Clin Proc. 20:241-248.) DHE differs from ergotamine in that it is less toxic, less emetic, devoid of uterotonic activity, and it displays decreased peripheral vasoconstrictor activity. DHE was used to terminate individual migraine attacks for the next 40 years. In 1986, Raskin described the use of DHE for the treatment of continuous migraines. (Raskin, N. H. 1986. Repetitive intravenous DHE as therapy for intractable migraine. Neurology. 36:995-997.).

Following the advent of the triptans, the use of DHE declined, due to the ease of use and improved side effect profiles of triptans. However, there remain many patients who respond poorly to triptans, but respond significantly better to DHE. Many patients taking triptans for acute migraine treatment discontinue use due to lack of efficacy, migraine recurrence, cost, and/or side effects. There remains a need for a convenient self-administrable form of DHE for individual patient use.

Currently available DHE products listed in the United States Food and Drug Administration's Orange Book of approved drug products include: D.H.E. 45, a 1.0 mL 1 mg/mL injectable formulation of DHE mesylate for treatment of migraine headaches with or without aura and the acute treatment of cluster headache episodes; Migranal®, a 0.5 mg/mL nasal spray formulation of DHE mesylate; and Embolex®, an injectable DHE mesylate formulation containing heparin sodium and lidocaine hydrochloride. No Orange Book product includes an injector with a formulation of more than about 1.0 mg/mL DHE mesylate.

Therefore, there is a need for stable, higher concentration DHE mesylate formulations for convenient, effective, and safe treatment of head pain, including ready-to-use pre-filled injectors of DHE mesylate.

### BRIEF SUMMARY OF THE INVENTION

### Pre-Filled DHE Mesylate Injector

The present disclosure provides a pre-filled injector comprising a pharmaceutically acceptable formulation of DHE mesylate at a concentration of about 3 mg/mL to about 6 mg/mL and carbon dioxide at a concentration sufficient to limit the oxidative degradation of DHE. In some aspects, the concentration of DHE mesylate in the pharmaceutically acceptable formulation is about 3.0 mg/mL, about 3.5 mg/mL, about 4.0 mg/mL, about 4.5 mg/mL, about 5.0 mg/mL, about 5.5 mg/mL, or about 6.0 mg/mL.

In some aspects, the pharmaceutically acceptable formulation further comprises caffeine.

In some aspects, the pharmaceutically acceptable formulation comprises caffeine at a concentration of about 5 mg/mL to about 15 mg/mL.

In some aspects, the pharmaceutically acceptable formulation comprises an osmotic agent.

In some aspects, the pharmaceutically acceptable formulation comprises an osmotic agent selected from the group consisting of dextrose, glycerin, mannitol, and sucrose, or combinations thereof.

In some aspects, the osmotic agent is dextrose at a concentration of about 1 mg/mL to about 20 mg/mL.

In some aspects, the osmotic agent is glycerin at a concentration of about 1 mg/mL to about 20 mg/mL.

In some aspects, the osmotic agent is mannitol at a concentration of about 20 mg/mL to about 50 mg/mL.

In some aspects, the osmotic agent is sucrose at a concentration of about 2 mg/mL to about 20 mg/mL.

In some aspects, the volume dispensed from the pre-filled injector can be about 0.10 mL, about 0.15 mL, about 0.20 mL, about 0.25 mL, or about 0.30 mL.

In some aspects, the pharmaceutically acceptable formulation further comprises an antioxidant.

In some aspects, the antioxidant is selected from the group consisting of: methionine, monothioglycerol, sodium bisulfite, sodium metabisulfite, sodium thiosulfate, sodium citrate, and thiourea, or combinations thereof.

In some aspects, the concentration of methionine is about 1.5 mg/mL to about 5 mg/mL.

In some aspects, the concentration of monothioglycerol is about 2 mg/mL to about 3 mg/mL.

In some aspects, the concentration of sodium citrate is about 0.1 mg/mL to about 4.0 mg/mL.

In some aspects, the pharmaceutically acceptable formulation is stored in a cartridge operably attached to the injector.

In some aspects, the volume of the cartridge containing the pharmaceutically acceptable formulation is about 3.0 mL.

In some aspects, the pH of the pharmaceutically acceptable formulation is between about 2.5 and about 4.5.

In some aspects, the pharmaceutically acceptable formulation further comprises a preservative.

In some aspects, the preservative is an isomer of cresol selected from the group consisting of o-cresol, m-cresol, and p-cresol, or combinations thereof. In some aspects, the preservative is benzyl alcohol. In some aspects, the preservative is a composition of one or more parabens. In some aspects, the composition of one or more parabens comprises methylparaben. In some aspects, the composition of one or more parabens comprises propylparaben. In some aspects, the composition of one or more parabens comprises methylparaben and propylparaben.

In some aspects, the preservative is benzyl alcohol. In some aspects, benzyl alcohol is present at a concentration of about 5 mg/mL to about 100 mg/mL.

In some aspects, the pharmaceutically acceptable formulation is stable.

In some aspects, the pharmaceutically acceptable formulation is substantially free of one or more of impurities selected from the group consisting of Impurity A, Impurity B, Impurity C, Impurity D, and Impurity E.

### DHE Mesylate Formulation

The present disclosure also provides a pharmaceutically acceptable formulation comprising DHE mesylate, a pharmaceutically acceptable alcohol, caffeine, an isomer of cresol (e.g., o-cresol, m-cresol, or p-cresol), carbon dioxide at a concentration sufficient to retard oxidative degradation of the DHE mesylate, and an osmotic agent.

In some aspects, the pharmaceutically acceptable formulation comprises about 3 mg/mL to about 5 mg/mL DHE mesylate.

In some aspects, the pharmaceutically acceptable formulation comprises a pharmaceutically acceptable alcohol selected from the group consisting of: propylene glycol, ethanol, and a polyethylene glycol, or combinations thereof.

In some aspects, the pharmaceutically acceptable formulation comprises a pharmaceutically acceptable alcohol at a concentration of about 3 mg/mL to about 10 mg/mL.

In some aspects, the pharmaceutically acceptable alcohol is ethanol.

In some aspects, the pharmaceutically acceptable formulation comprises about 5 mg/mL to about 15 mg/mL caffeine.

In some aspects, the pharmaceutically acceptable formulation comprises about 1 mg/mL to about 3 mg/mL an isomer of cresol (e.g., o-cresol, m-cresol, or p-cresol).

In some aspects, the pharmaceutically acceptable formulation comprises carbon dioxide at a concentration sufficient to retard oxidative degradation of the DHE mesylate.

In some aspects, the pharmaceutically acceptable formulation comprises an osmotic agent selected from the group consisting of dextrose, glycerin, mannitol, and sucrose, or combinations thereof.

In some aspects, the osmotic agent is dextrose at a concentration of about 1 mg/mL to about 20 mg/mL.

In some aspects, the osmotic agent is glycerin at a concentration of about 1 mg/mL to about 20 mg/mL.

In some aspects, the osmotic agent is mannitol at a concentration of about 20 mg/mL to about 50 mg/mL.

In some aspects, the osmotic agent is sucrose at a concentration of about 2 mg/mL to about 20 mg/mL.

In some aspects, the pharmaceutically acceptable formulation further comprises an antioxidant.

In some aspects, the pharmaceutically acceptable formulation comprises an antioxidant selected from the group consisting of methionine, monothioglycerol, sodium bisulfite, sodium metabisulfite, sodium thiosulfate, sodium citrate, and thiourea, or combinations thereof.

In some aspects, the concentration of methionine is about 1.5 mg/mL to about 5 mg/mL.

In some aspects, the concentration of monothioglycerol is about 2 mg/mL to about 3 mg/mL.

In some aspects, the concentration of sodium metabisulfite is about 0.2 mg/mL to about 4.0 mg/mL.

In some aspects, the concentration of sodium citrate is about 0.1 mg/mL to about 4.0 mg/mL.

In some aspects, the pharmaceutically acceptable formulation further comprises a cyclodextrin.

In some aspects, the pharmaceutically acceptable formulation comprises a cyclodextrin selected from the group consisting of 2-hydroxypropyl-β-cyclodextrin, O-methyl-β-cyclodextrin, and γ-cyclodextrin, or combinations thereof.

In some aspects, an injector comprises the pharmaceutically acceptable formulation.

In some aspects, an injector can be adjusted to dispense about 0.1 mL to about 0.3 mL of the pharmaceutically acceptable formulation.

In some aspects, an injector can be adjusted to dispense about 0.10 mL, about 0.15 mL, about 0.20 mL, about 0.25 mL, or about 0.30 mL of the pharmaceutically acceptable formulation.

In some aspects, the pharmaceutically acceptable formulation is contained within a sterilized cartridge with a capacity of about 3.0 mL, which can be operably attached to an injector to dispense a quantity of the pharmaceutically acceptable formulation.

In some aspects, the pharmaceutically acceptable formulation is stable.

In some aspects, the pharmaceutically acceptable formulation is substantially free of one or more of impurities selected from the group consisting of Impurity A, Impurity B, Impurity C, Impurity D, and Impurity E.

### Method of Treatment

The present disclosure also provides a method of treating a migraine or a cluster headache attack in a patient, the method comprising administering to a patient in need thereof a therapeutically effective amount of a pharmaceutically acceptable formulation, the formulation comprising DHE mesylate at a concentration of about 3 mg/mL to about 6 mg/mL, and carbon dioxide at a concentration sufficient to retard oxidative degradation of DHE mesylate.

In some aspects, the pharmaceutically acceptable formulation further comprises caffeine.

In some aspects, the caffeine in the pharmaceutically acceptable formulation is at a concentration of about 5 mg/mL to about 15 mg/mL.

In some aspects, the pharmaceutically acceptable formulation further comprises an osmotic agent.

In some aspects, the pharmaceutically acceptable formulation comprises an osmotic agent selected from the group consisting of: dextrose, glycerin, mannitol, and sucrose, or combinations thereof.

In some aspects, the osmotic agent is dextrose at a concentration of about 1 mg/mL to about 20 mg/mL.

In some aspects, the osmotic agent is glycerin at a concentration of about 1 mg/mL to about 20 mg/mL.

In some aspects, the osmotic agent is mannitol at a concentration of about 20 mg/mL to about 50 mg/mL.

In some aspects, the osmotic agent is sucrose at a concentration of about 2 mg/mL to about 20 mg/mL.

In some aspects, the pharmaceutically acceptable formulation further comprises an antioxidant.

In some aspects, the pharmaceutically acceptable formulation comprises an antioxidant selected from the group consisting of: methionine, monothioglycerol, sodium bisulfite, sodium metabisulfite, sodium thiosulfate, sodium citrate, and thiourea, or combinations thereof.

In some aspects, the concentration of methionine in the pharmaceutically acceptable formulation is about 1.5 mg/mL to about 5 mg/mL.

In some aspects, the concentration of monothioglycerol in the pharmaceutically acceptable formulation is about 2 mg/mL to about 3 mg/mL.

In some aspects, the concentration of sodium metabisulfite in the pharmaceutically acceptable formulation is about 0.2 mg/mL to about 4.0 mg/mL.

In some aspects, the concentration of sodium citrate in the pharmaceutically acceptable formulation is about 0.1 mg/mL to about 4.0 mg/mL.

In some aspects, the pharmaceutically acceptable formulation further comprises a cyclodextrin.

In some aspects, the cyclodextrin in the pharmaceutically acceptable formulation is selected from the group consisting of: 2-hydroxypropyl-β-cyclodextrin, O-methyl-β-cyclodextrin, and γ-cyclodextrin, or combinations thereof.

In some aspects, the pharmaceutically acceptable formulation further comprises a preservative.

In some aspects, the preservative is an isomer of cresol selected from the group consisting of o-cresol, m-cresol, and p-cresol, or combinations thereof. In some aspects, the preservative is benzyl alcohol. In some aspects, the preservative is a composition of one or more parabens. In some aspects, the composition of one or more parabens comprises methylparaben. In some aspects, the composition of one or more parabens comprises propylparaben. In some aspects, the composition of one or more parabens comprises methylparaben and propylparaben.

In some aspects, the preservative is benzyl alcohol. In some aspects, benzyl alcohol is present at a concentration of about 5 mg/mL to about 100 mg/mL.

In some aspects, the method of treatment further comprises subcutaneous administration of the pharmaceutically acceptable formulation to the patient.

In some aspects, the method of treatment further comprises intramuscular administration of the pharmaceutically acceptable formulation to the patient.

In some aspects, the method of treatment further comprises intravenous administration of the pharmaceutically acceptable formulation to the patient.

In some aspects, the method of treatment comprises administering the pharmaceutically acceptable formulation to the patient from an injector pre-loaded with the pharmaceutically acceptable formulation.

In some aspects, the method of treatment further comprises administration of about 0.1 to about 0.3 mL of the pharmaceutically acceptable formulation.

In some aspects, the pharmaceutically acceptable formulation is stable.

In some aspects, the pharmaceutically acceptable formulation is substantially free of one or more of impurities selected from the group consisting of Impurity A, Impurity B, Impurity C, Impurity D, and Impurity E.

### Pre-Filled Injector Method of Manufacture

The present disclosure further provides a method of manufacturing a pre-filled injector comprising a pharmaceutically acceptable formulation comprising about 3 mg/mL to about 6 mg/mL DHE mesylate, about 3 mg/mL to about 10 mg/mL of a pharmaceutically acceptable alcohol, about 5 mg/mL to about 15 mg/mL caffeine, about 1 mg/mL to about 3 mg/mL of an isomer of cresol (e.g., o-cresol, m-cresol, or p-cresol), and an osmotic agent; sparging the pharmaceutically acceptable formulation with carbon dioxide at a concentration sufficient to retard oxidative degradation of the DHE mesylate; loading a sterile cartridge with the pharmaceutically acceptable formulation; and attaching the sterile cartridge comprising the pharmaceutically acceptable formulation operably to an injector.

In some aspects, the pharmaceutically acceptable alcohol is selected from the group consisting of: propylene glycol, ethanol, and a polyethylene glycol, or combinations thereof.

In some aspects, the pharmaceutically acceptable alcohol is ethanol.

In some aspects, the cartridge has a capacity of about 3.0 mL.

In some aspects, the osmotic agent is dextrose at a concentration of about 1 mg/mL to about 20 mg/mL.

In some aspects, the osmotic agent is glycerin at a concentration of about 1 mg/mL to about 20 mg/mL.

In some aspects, the osmotic agent is mannitol at a concentration of about 20 mg/mL to about 50 mg/mL.

In some aspects, the osmotic agent is sucrose at a concentration of about 2 mg/mL to about 20 mg/mL.

In some aspects, the pharmaceutically acceptable formulation further comprises a cyclodextrin.

In some aspects, the pharmaceutically acceptable formulation comprises a cyclodextrin selected from the group consisting of: 2-hydroxypropyl-β-cyclodextrin, O-methyl-β-cyclodextrin, and γ-cyclodextrin, or combinations thereof.

In some aspects, the pharmaceutically acceptable formulation further comprises an antioxidant.

In some aspects, the pharmaceutically acceptable formulation comprises an antioxidant selected from the group consisting of: methionine, monothioglycerol, sodium bisulfite, sodium metabisulfite, sodium thiosulfate, sodium citrate, and thiourea, or combinations thereof.

In some aspects, the concentration of methionine is about 1.5 mg/mL to about 5 mg/mL.

In some aspects, the concentration of monothioglycerol is about 2 mg/mL to about 3 mg/mL.

In some aspects, the concentration of sodium metabisulfite is about 0.2 mg/mL to about 4.0 mg/mL.

In some aspects, the concentration of sodium citrate is about 0.1 mg/mL to about 4.0 mg/mL.

In some aspects, the method of manufacture of the injector comprises inclusion in the injector of an adjustable input mechanism, such that the volume to be dispensed from the injector is adjustable to a volume of about 0.1 mL to about 3.0 mL.

In some aspects, the pharmaceutically acceptable formulation is stable.

In some aspects, the pharmaceutically acceptable formulation is substantially free of one or more of impurities selected from the group consisting of Impurity A, Impurity B, Impurity C, Impurity D, and Impurity E.

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient.

The term “unit dosage form” or “unit dose composition” as used herein refers to a device containing a quantity of the therapeutic compound, said quantity being such that one or more predetermined units may be provided as a single therapeutic administration.

The term “Cₘₐₓ” as used herein refers to the maximum plasma concentration of a drug after administration of the drug.

The term “Tₘₐₓ” as used herein refers to the time required to reach the maximal plasma concentration Cₘₐₓ after administration of a drug.

The term “AUC” as used herein refers to the area under the curve of a plot of plasma concentration versus time following administration of a drug.

The term “AUC₀₋ₜ” as used herein refers to the area under the drug concentration-time curve from time zero to the time of the last measurable concentration (Cₜ).

The term “AUC_{0-∞}” as used herein refers to the area under the drug concentration-time curve from time zero to infinity.

The term “steady state” as used herein means that the amount of the drug reaching the system is approximately the same as the amount of the drug leaving the system. Thus, at “steady-state,” the patient's body eliminates the drug at approximately the same rate that the drug becomes available to the patient's system through absorption into the blood stream.

The term “mean” refers to an average value in a patient population. For example, a “mean Cₘₐₓ” refers to an average of the maximum plasma concentrations of a drug in a patient population.

The term “treating” or “treatment” as used herein refers to the administration of a composition to a subject for therapeutic purposes.

The term “serum concentration” generally refers to the amount of a drug or other compound in the circulation, both bound to proteins and unbound, the latter of which generally corresponds to the therapeutically active fraction.

The term “bioavailability” generally refers to the rate and extent to which the active ingredient is absorbed from a drug product and becomes available at the site of action.

“Bioequivalence” is a term in pharmacokinetics generally used to assess the expected in vivo biological equivalence of two proprietary preparations of a drug. Two pharmaceutical products are bioequivalent if they are pharmaceutically equivalent and their bioavailabilities (rate and extent of availability) after administration in the same molar dose are similar to such a degree that their effects, with respect to both efficacy and safety, can be expected to be essentially the same.

The term “stable” as used herein refers to a composition characterized by one or more substantially unchanged characteristics after storage for 18 months or more in a sealed container at 40° C., the one or more substantially unchanged characteristics selected from the group consisting of: the concentration of DHE in the pharmaceutical composition; and the concentration of one or more impurities in the pharmaceutical composition (e.g., Impurity A, Impurity B, Impurity C, Impurity D, and/or Impurity E). In some aspects, a pharmaceutically acceptable formulation described herein is stable for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

The term “substantially free of” refers to a composition having less than 0.1% w/w, 0.2% w/w, 0.3% w/w, 0.4% w/w, 0.5% w/w, 0.6% w/w, 0.7% w/w, 0.8% w/w, 0.9% w/w, 1.0% w/w, 1.1% w/w, 1.2% w/w, 1.3% w/w, 1.4% w/w, 1.5% w/w, 1.6% w/w, 1.7% w/w, 1.8% w/w, 1.9% w/w, or 2.0% w/w of a specified component (e.g., Imp-A, Imp-B, Imp-C, Imp-D, or Imp-E).

The term “injector” as used herein refers to an apparatus wherein an individual can administer a formulation, such as a pharmaceutical formulation, to oneself. In some aspects, the injector delivers a single dose. In some aspects, the injector is adjustable to deliver various volumes of the DHE formulation. In some aspects, multiple injections can be dispensed from the same injector. In other aspects, part or all of the injector is disposable and/or reusable. In some aspects, part or all of the injector is opaque, and in further specific embodiments at least one part of the injector that is opaque is the part that houses the pharmaceutical formulation. An injector may be supplied separately from the pharmaceutical formulations, in alternative aspects. The injector may comprise an exchangeable vessel for replacing the pharmaceutical formulation, such as an insert, cartridge, vial, and so forth. Such an exchangeable vessel may be glass or plastic, for example. It will be understood by the skilled artisan that the injector may be an autoinjector, a pen injector, a needle-less injector, or any other injection device suitable for the delivery of a pharmaceutical formulation.

The term “DHE” refers to dihydroergotamine. DHE is also known as (5′α)-9,10-dihydro-12′-hydroxy-2′-methyl-5′-(phenylmethyl)-ergotaman-3′,6′,18-trione. DHE has the following chemical structure:

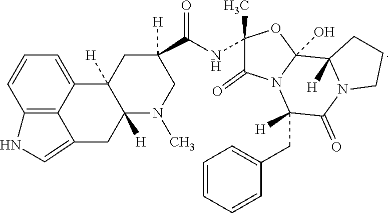

The term “DHE mesylate” refers to the mesylate salt of dihydroergotamine. DHE mesylate is known chemically as ergotaman-3′,6′,19-trione, 9,10-dihydro-12′-hydroxy-2′-methyl-5′-(phenylmethyl)-, (5′α)-, monomethane-sulfonate. Its molecular weight is 679.78 and its empirical formula is C₃₃H₃₇N₅O₅.CH₄O₃S. DHE mesylate has the following chemical structure:

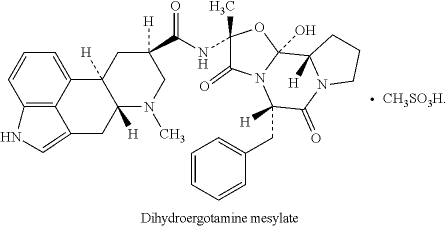

The terms “Impurity A” and “Imp-A” refer to the chemical entity having the following structure:

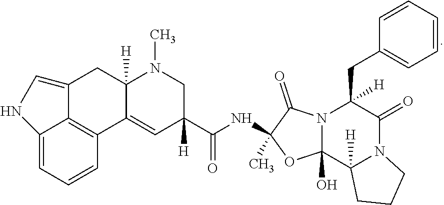

The terms “Impurity B” and “Imp-B” refer to the chemical entity having the following structure:

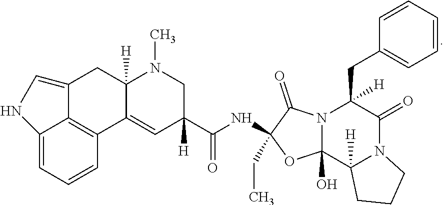

The terms “Impurity C” and “Imp-C” refer to the chemical entity having the following structure:

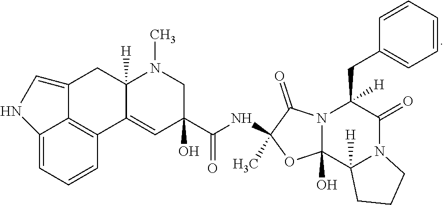

The terms “Impurity D” and “Imp-D” refer to the chemical entity having the following structure:

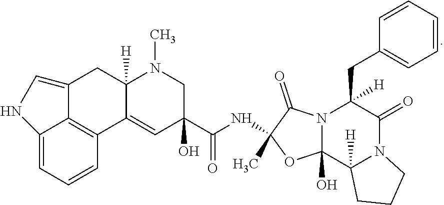

The terms “Impurity E” and “Imp-E” refer to the chemical entity having the following structure:

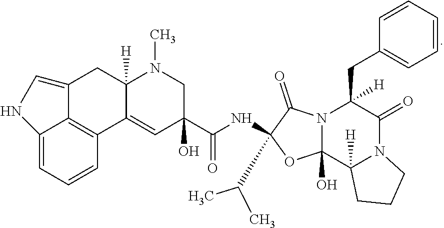

The term “Water for Injection” (“WFI”) refers to water that meets the U.S.P. requirements (or foreign equivalent) for “Water for Injection.” These requirements include bacterial endotoxins of not more than 0.25 U.S.P. EU per mL, total organic carbon (TOC) content of <500 parts per billion (ppb), and conductivity of 1.3 μS/cm. Water for Injection also includes compendial and non-compendial water classifications that meet the requirements of U.S.P. Water for Injection. Examples include water labeled or marketed as “Low Endotoxin U.S.P. Purified Water” and “WFI Quality Water.”

The term “osmotic agent” as used herein, refers to compounds capable of imbibing water and can thereby establish an osmotic pressure gradient across an adjacent semipermeable barrier. Osmotic agents are also referred to as “osmogens” or “osmagents”. Osmotic agents include, but are not limited to: glycerin, sodium chloride, magnesium chloride, calcium chloride, potassium chloride, magnesium sulfate, potassium sulfate, sodium carbonate, sodium sulfate, ascorbic acid, benzoic acid, fumaric acid, citric acid, mannitol, sucrose, sorbitol, xylitol, lactose, dextrose, and trehalose.

### Pre-Filled DHE Mesylate Injector

The present disclosure provides a pre-filled injector comprising a pharmaceutically acceptable formulation of DHE mesylate at a concentration of about 0.5 mg/mL to about 9.0 mg/mL and carbon dioxide at a concentration sufficient to limit the oxidative degradation of DHE.

In some aspects, the concentration of DHE mesylate in the pharmaceutically acceptable formulation is about 1.0 mg/mL to about 8.0 mg/mL.

In some aspects, the concentration of DHE mesylate in the pharmaceutically acceptable formulation is about 2.0 mg/mL to about 7.0 mg/mL.

In some aspects, the concentration of DHE mesylate in the pharmaceutically acceptable formulation is about 3.0 mg/mL to about 6.0 mg/mL. In some aspects, the concentration of DHE mesylate in the pharmaceutically acceptable formulation is about 3.0 mg/mL, about 3.5 mg/mL, about 4.0 mg/mL, about 4.5 mg/mL, about 5.0 mg/mL, about 5.5 mg/mL, or about 6.0 mg/mL.

In some aspects, the concentration of DHE mesylate in the pharmaceutically acceptable formulation is about 3.5 mg/mL to about 5.5 mg/mL.

In some aspects, the concentration of DHE mesylate in the pharmaceutically acceptable formulation is about 4.0 mg/mL to about 5.0 mg/mL.

In some aspects, the pharmaceutically acceptable formulation further comprises caffeine.

In some aspects, the pharmaceutically acceptable formulation comprises caffeine at a concentration of about 1 mg/mL to about 20 mg/mL.

In some aspects, the pharmaceutically acceptable formulation comprises caffeine at a concentration of about 2 mg/mL to about 18 mg/mL.

In some aspects, the pharmaceutically acceptable formulation comprises caffeine at a concentration of about 4 mg/mL to about 16 mg/mL.

In some aspects, the pharmaceutically acceptable formulation comprises caffeine at a concentration of about 5 mg/mL to about 15 mg/mL. In some aspects, the pharmaceutically acceptable formulation comprises caffeine at a concentration of about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, or about 15 mg/mL.

In some aspects, the pharmaceutically acceptable formulation comprises caffeine at a concentration of about 6 mg/mL to about 14 mg/mL.

In some aspects, the pharmaceutically acceptable formulation comprises caffeine at a concentration of about 7 mg/mL to about 13 mg/mL.

In some aspects, the pharmaceutically acceptable formulation in the pre-filled injector comprises an osmotic agent.

In some aspects, the pharmaceutically acceptable formulation in the pre-filled injector comprises an osmotic agent selected from the group consisting of dextrose, glycerin, mannitol, and sucrose, or combinations thereof.

In some aspects, the osmotic agent in the pharmaceutically acceptable formulation in the pre-filled injector is dextrose.

In some aspects, the osmotic agent in the pharmaceutically acceptable formulation in the pre-filled injector is dextrose at a concentration of about 0.1 mg/mL to about 25 mg/mL.

In some aspects, the osmotic agent in the pharmaceutically acceptable formulation in the pre-filled injector is dextrose at a concentration of about 1 mg/mL to about 20 mg/mL.

In some aspects, the osmotic agent in the pharmaceutically acceptable formulation in the pre-filled injector is dextrose at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation in the pre-filled injector is dextrose at a concentration of about 3 mg/mL to about 18 mg/mL.

In some aspects, the osmotic agent in the pharmaceutically acceptable formulation in the pre-filled injector is dextrose at a concentration of about 5 mg/mL to about 16 mg/mL.

In some aspects, the osmotic agent in the pharmaceutically acceptable formulation in the pre-filled injector is glycerin.

In some aspects, the osmotic agent in the pharmaceutically acceptable formulation in the pre-filled injector is glycerin at a concentration of about 0.1 mg/mL to about 25 mg/mL.

In some aspects, the osmotic agent in the pharmaceutically acceptable formulation in the pre-filled injector is glycerin at a concentration of about 1 mg/mL to about 20 mg/mL.

In some aspects, the osmotic agent in the pharmaceutically acceptable formulation in the pre-filled injector is glycerin at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation in the pre-filled injector is glycerin at a concentration of about 3 mg/mL to about 18 mg/mL.

In some aspects, the osmotic agent in the pharmaceutically acceptable formulation in the pre-filled injector is glycerin at a concentration of about 5 mg/mL to about 16 mg/mL.

In some aspects, the osmotic agent in the pharmaceutically acceptable formulation in the pre-filled injector is mannitol.

In some aspects, the osmotic agent in the pharmaceutically acceptable formulation in the pre-filled injector is mannitol at a concentration of about 15 mg/mL to about 55 mg/mL.

In some aspects, the osmotic agent in the pharmaceutically acceptable formulation in the pre-filled injector is mannitol at a concentration of about 20 mg/mL to about 50 mg/mL. In some aspects, the osmotic agent in the pharmaceutically acceptable formulation in the pre-filled injector is mannitol at a concentration of about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL.

In some aspects, the osmotic agent in the pharmaceutically acceptable formulation in the pre-filled injector is mannitol at a concentration of about 25 mg/mL to about 45 mg/mL.

In some aspects, the osmotic agent in the pharmaceutically acceptable formulation in the pre-filled injector is mannitol at a concentration of about 30 mg/mL to about 40 mg/mL. In some aspects, the osmotic agent in the pharmaceutically acceptable formulation in the pre-filled injector is sucrose.

In some aspects, the osmotic agent in the pharmaceutically acceptable formulation in the pre-filled injector is sucrose at a concentration of about 0.5 mg/mL to about 25 mg/mL.

In some aspects, the osmotic agent in the pharmaceutically acceptable formulation in the pre-filled injector is sucrose at a concentration of about 2 mg/mL to about 20 mg/mL.

In some aspects, the osmotic agent in the pharmaceutically acceptable formulation in the pre-filled injector is sucrose at a concentration of about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation in the pre-filled injector is sucrose at a concentration of about 4 mg/mL to about 18 mg/mL.

In some aspects, the osmotic agent in the pharmaceutically acceptable formulation in the pre-filled injector is sucrose at a concentration of about 6 mg/mL to about 16 mg/mL.

In some aspects, the volume dispensed from the pre-filled injector is about 0.05 mL to about 0.5 mL per injection.

In some aspects, the volume dispensed from the pre-filled injector is about 0.1 mL to about 0.4 mL per injection.

In some aspects, the volume dispensed from the pre-filled injector is about 0.1 mL to about 0.3 mL per injection.

In some aspects, the volume dispensed from the pre-filled injector can be about 0.10 mL, about 0.15 mL, about 0.20 mL, about 0.25 mL, or about 0.30 mL.

In some aspects, the volume dispensed from the pre-filled injector is about 0.13 mL to about 0.27 mL per injection.

In some aspects, the volume dispensed from the pre-filled injector is about 0.15 mL to about 0.25 mL per injection.

In some aspects, the pharmaceutically acceptable formulation in the pre-filled injector further comprises an antioxidant.

In some aspects, the antioxidant in the pharmaceutically acceptable formulation in the pre-filled injector is methionine. In some aspects, the concentration of methionine in the pharmaceutically acceptable formulation is about 1.5 mg/mL to about 5 mg/mL. In some aspects, the concentration of methionine in the pharmaceutically acceptable formulation is about 1.5 mg/mL, about 2 mg/mL, about 2.5 mg/mL, about 3 mg/mL, about 3.5 mg/mL, about 4 mg/mL, about 4.5 mg/mL, or about 5 mg/mL.

In some aspects, the antioxidant in the pharmaceutically acceptable formulation in the pre-filled injector is monothioglycerol. In some aspects, the concentration of monothioglycerol in the pharmaceutically acceptable formulation is about 2 mg/mL to about 3 mg/mL. In some aspects, the concentration of monothioglycerol in the pharmaceutically acceptable formulation is about 2 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, or about 3 mg/mL.

In some aspects, the antioxidant in the pharmaceutically acceptable formulation in the pre-filled injector is sodium bisulfite.

In some aspects, the antioxidant in the pharmaceutically acceptable formulation in the pre-filled injector is sodium metabisulfite.

In some aspects, the concentration of sodium metabisulfite in the pharmaceutically acceptable formulation is about 0.1 mg/mL to about 5.0 mg/mL.

In some aspects, the concentration of sodium metabisulfite in the pharmaceutically acceptable formulation is about 0.2 mg/mL to about 4.0 mg/mL.

In some aspects, the concentration of sodium metabisulfite in the pharmaceutically acceptable formulation is about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium metabisulfite in the pharmaceutically acceptable formulation is about 0.5 mg/mL to about 3.5 mg/mL.

In some aspects, the concentration of sodium metabisulfite in the pharmaceutically acceptable formulation is about 1.0 mg/mL to about 3.0 mg/mL.

In some aspects, the antioxidant in the pharmaceutically acceptable formulation in the pre-filled injector is sodium thiosulfate.

In some aspects, the antioxidant in the pharmaceutically acceptable formulation in the pre-filled injector is sodium citrate.

In some aspects, the concentration of sodium citrate in the pharmaceutically acceptable formulation is about 0.05 mg/mL to about 5.0 mg/mL.

In some aspects, the concentration of sodium citrate in the pharmaceutically acceptable formulation is about 0.1 mg/mL to about 4.0 mg/mL.

In some aspects, the concentration of sodium citrate in the pharmaceutically acceptable formulation is about 0.1 mg/mL, about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium citrate in the pharmaceutically acceptable formulation is about 0.5 mg/mL to about 3.5 mg/mL.

In some aspects, the concentration of sodium citrate in the pharmaceutically acceptable formulation is about 1.0 mg/mL to about 3.0 mg/mL.

In some aspects, the antioxidant in the pharmaceutically acceptable formulation in the pre-filled injector is thiourea.

In some aspects, the pharmaceutically acceptable formulation further comprises a preservative.

In some aspects, the preservative is an isomer of cresol selected from the group consisting of o-cresol, m-cresol, and p-cresol, or combinations thereof. In some aspects, the preservative is o-cresol. In some aspects, the preservative is p-cresol.

In some aspects, the preservative is m-cresol. In some aspects, m-cresol is present at a concentration of about 1 mg/mL to about 5 mg/mL. In some aspects, m-cresol is present at a concentration of about 1 mg/mL to about 2.5 mg/mL. In some aspects, m-cresol is present at a concentration of about 1.5 mg/mL.

In some aspects, the preservative is benzyl alcohol. In some aspects, benzyl alcohol is present at a concentration of about 5 mg/mL to about 100 mg/mL. In some aspects, benzyl alcohol is present at a concentration of about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 18 mg/mL, about 19 mg/mL, about 20 mg/mL, about 25 mg/mL, about 30 mg/mL, about 35 mg/mL, about 40 mg/mL, about 50 mg/mL, about 60 mg/mL, about 70 mg/mL, about 80 mg/mL, about 90 mg/mL, or about 100 mg/mL. In some aspects, benzyl alcohol is present at a concentration of about 10 mg/mL.

In some aspects, the preservative is a composition of one or more parabens. In some aspects, the composition of one or more parabens comprises methylparaben. In some aspects, the composition of one or more parabens comprises propylparaben. In some aspects, the composition of one or more parabens comprises methylparaben and propylparaben. In some aspects, the pharmaceutically acceptable formulation is stored in a cartridge operably attached to the injector.

In some aspects, the capacity of the cartridge containing the pharmaceutically acceptable formulation is about 3.0 mL. In some aspects, the pH of the pharmaceutically acceptable formulation is between about 2.5 and about 4.5.

In some aspects, the pH of the pharmaceutically acceptable formulation is adjusted to a target range by adding a quantity of carbon dioxide as a buffering agent.

In some aspects, the pharmaceutically acceptable formulation is sparged with carbon dioxide at a concentration sufficient to retard the oxidation of the dihydroergotamine mesylate.

In some aspects, the pharmaceutically acceptable formulation is contained within a sterilized cartridge at a concentration sufficient to retard the oxidation of the dihydroergotamine mesylate.

In some aspects, the volume dispensed by the pre-filled injector is adjustable.

In some aspects, the volume dispensed by the pre-filled injector is not adjustable.

In some aspects, the same injector may dispense a quantity of the pharmaceutically acceptable formulation multiple times before the volume of the pharmaceutically acceptable formulation contained within the pre-filled injector is depleted.

In some aspects, the pharmaceutically acceptable formulation is stored in a cartridge operably attached to the injector.

In some aspects, the cartridge containing the pharmaceutically acceptable formulation is disposable.

In some aspects, the cartridge is sterile prior to loading with the pharmaceutically acceptable formulation.

In some aspects, a first cartridge containing the pharmaceutically acceptable formulation and operably attached to the injector can be replaced with a second cartridge, such that the injector dispenses the contents of the second cartridge.

In some aspects, the preparation of the pharmaceutically acceptable formulation in the pre-filled injector comprises adjustment of the pharmaceutically acceptable formulation to a target pH of about 2.5 and about 4.5 with methanesulfonic acid, carbon dioxide, and/or sodium hydroxide.

In some aspects, the pre-filled injector is a fixed dose pen injector.

In some aspects, the pre-filled injector is a fixed dose needle-less pen injector.

In some aspects, the pre-filled injector is a pen injector with an adjustable dispensed volume.

In some aspects, the pre-filled injector is a needle-less pen injector with an adjustable dispensed volume.

In some aspects, the pre-filled injector can dispense about 10 injections per cartridge.

In some aspects, the formulations of the present disclosure are compatible with subcutaneous, intravenous, intradermal, or intramuscular administration (e.g., by injection or by infusion).

In some aspects, the pharmaceutically acceptable formulation of the present disclosure is administered by injection using any suitable device. For example, a pharmaceutically acceptable formulation of the present disclosure can be placed into a syringe, a pen injection device, or an autoinjector. Common injectors range from a simple manual syringe system to an autoinjector. A manual syringe system would include a syringe comprising a barrel and a plunger and an appropriately-sized needle. Such simple syringes may be adapted to accept pre-filled cartridges, be packaged with the drug formulation loaded in the syringe, or used with vials, for example. Formulated drugs such as DHE mesylate may be prepared and filled into ampoules, prefilled cartridges, syringes, or vials that may be single or multi-dose containers, for example.

In some aspects, the injector is a multi-dose injector pump or a multi-dose injector. The formulation is presented in the injector in such a fashion that the formulation is readily able to flow out of the needle upon actuation of an injector, in order to deliver the DHE formulations. Suitable injectors include, but are not limited to, pen injectors and autoinjectors manufactured by Becton-Dickenson, Swedish Healthcare Limited (SHL Group), YpsoMed Ag, and the like.

In some aspects, the pharmaceutically acceptable formulation of the present disclosure is provided ready for administration in a vial, a cartridge, or a pre-filled syringe.

In some aspects, the pharmaceutically acceptable formulation is stable.

In some aspects, the pharmaceutically acceptable formulation is stable as determined by a substantially unchanged amount of DHE following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the pharmaceutically acceptable formulation is stable as determined by a substantially unchanged amount of one or more impurities following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the one or more impurities comprise Impurity A. In some aspects, the one or more impurities comprise Impurity B. In some aspects, the one or more impurities comprise Impurity C. In some aspects, the one or more impurities comprise Impurity D. In some aspects, the one or more impurities comprise Impurity E. In some aspects, the one or more impurities comprise Impurity A-E.

In some aspects, the pharmaceutically acceptable formulation is substantially free of one or more of impurities selected from the group consisting of Impurity A, Impurity B, Impurity C, Impurity D, and Impurity E.

### DHE Mesylate Formulation

The present disclosure also provides a pharmaceutically acceptable formulation comprising DHE mesylate, a pharmaceutically acceptable alcohol, caffeine, an isomer of cresol (e.g., o-cresol, m-cresol, or p-cresol), carbon dioxide at a concentration sufficient to retard oxidative degradation of the DHE mesylate, and an osmotic agent.

In some aspects, the pharmaceutically acceptable formulation comprises about 0.5 mg/mL to about 9.0 mg/mL DHE mesylate.

In some aspects, the pharmaceutically acceptable formulation comprises about 1.0 mg/mL to about 8.0 mg/mL DHE mesylate.

In some aspects, the pharmaceutically acceptable formulation comprises about 2.0 mg/mL to about 7.0 mg/mL DHE mesylate. In some aspects, the pharmaceutically acceptable formulation comprises about 3.0 mg/mL to about 6.0 mg/mL DHE mesylate. In some aspects, the pharmaceutically acceptable formulation comprises DHE mesylate at a concentration of about 3.0 mg/mL, about 3.5 mg/mL, about 4.0 mg/mL, about 4.5 mg/mL, about 5.0 mg/mL, about 5.5 mg/mL, or about 6.0 mg/mL.

In some aspects, the pharmaceutically acceptable formulation comprises DHE mesylate at a concentration of about 3.5 mg/mL to about 5.5 mg/mL.

In some aspects, the pharmaceutically acceptable formulation comprises DHE mesylate at a concentration of about 4.0 mg/mL to about 5.0 mg/mL.

In some aspects, the pharmaceutically acceptable formulation comprises a pharmaceutically acceptable alcohol.

In some aspects, the pharmaceutically acceptable formulation comprises a pharmaceutically acceptable alcohol selected from the group consisting of: propylene glycol, ethanol, and a polyethylene glycol, or combinations thereof.

In some aspects, the pharmaceutically acceptable formulation comprises a pharmaceutically acceptable alcohol at a concentration of about 3 mg/mL to about 10 mg/mL.

In some aspects, the pharmaceutically acceptable alcohol is ethanol.

In some aspects, the pharmaceutically acceptable formulation comprises caffeine at a concentration of about 1 mg/mL to about 20 mg/mL. In some aspects, the pharmaceutically acceptable formulation comprises about 5 mg/mL to about 15 mg/mL caffeine. In some aspects, the pharmaceutically acceptable formulation comprises caffeine at a concentration of about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, or about 15 mg/mL.

In some aspects, the pharmaceutically acceptable formulation comprises caffeine at a concentration of about 6 mg/mL to about 14 mg/mL.

In some aspects, the pharmaceutically acceptable formulation comprises caffeine at a concentration of about 7 mg/mL to about 13 mg/mL.

In some aspects, the pharmaceutically acceptable formulation comprises about 0.1 mg/mL to about 5 mg/mL of an isomer of cresol (e.g., o-cresol, m-cresol, or p-cresol).

In some aspects, the pharmaceutically acceptable formulation comprises about 1 mg/mL to about 3 mg/mL of an isomer of cresol (e.g., o-cresol, m-cresol, or p-cresol).

In some aspects, the pharmaceutically acceptable formulation comprises an isomer of cresol (e.g., o-cresol, m-cresol, or p-cresol) at a concentration of about 1.0 mg/mL, about 1.5 mg/mL, about 2.0 mg/mL, about 2.5 mg/mL, or about 3.0 mg/mL.

In some aspects, the pharmaceutically acceptable formulation comprises an isomer of cresol (e.g., o-cresol, m-cresol, or p-cresol) at a concentration of about 1.25 mg/mL to about 2.75 mg/mL.

In some aspects, the pharmaceutically acceptable formulation comprises an isomer of cresol (e.g., o-cresol, m-cresol, or p-cresol) at a concentration of about 1.5 mg/mL to about 2.5 mg/mL.

In some aspects, the pharmaceutically acceptable formulation comprises carbon dioxide at a concentration sufficient to retard oxidative degradation of the DHE mesylate.

In some aspects, the pharmaceutically acceptable formulation is sparged with carbon dioxide to achieve a chosen concentration of carbon dioxide.

In some aspects, the pharmaceutically acceptable formulation comprises an osmotic agent.

In some aspects, the pharmaceutically acceptable formulation comprises an osmotic agent selected from the group consisting of dextrose, glycerin, mannitol, and sucrose, or combinations thereof.

In some aspects, the osmotic agent in the pharmaceutically acceptable formulation is dextrose.

In some aspects, the osmotic agent in the pharmaceutically acceptable formulation is dextrose at a concentration of about 0.1 mg/mL to about 25 mg/mL.

In some aspects, the osmotic agent in the pharmaceutically acceptable formulation is dextrose at a concentration of about 1 mg/mL to about 20 mg/mL.

In some aspects, the osmotic agent in the pharmaceutically acceptable formulation is dextrose at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation is dextrose at a concentration of about 3 mg/mL to about 18 mg/mL.

In some aspects, the osmotic agent in the pharmaceutically acceptable formulation is dextrose at a concentration of about 5 mg/mL to about 16 mg/mL.

In some aspects, the osmotic agent in the pharmaceutically acceptable formulation is glycerin.

In some aspects, the osmotic agent in the pharmaceutically acceptable formulation is glycerin at a concentration of about 0.1 mg/mL to about 25 mg/mL.

In some aspects, the osmotic agent in the pharmaceutically acceptable formulation is glycerin at a concentration of about 1 mg/mL to about 20 mg/mL.

In some aspects, the osmotic agent in the pharmaceutically acceptable formulation is glycerin at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation is glycerin at a concentration of about 3 mg/mL to about 18 mg/mL.

In some aspects, the osmotic agent in the pharmaceutically acceptable formulation is glycerin at a concentration of about 5 mg/mL to about 16 mg/mL.

In some aspects, the osmotic agent in the pharmaceutically acceptable formulation is mannitol.

In some aspects, the osmotic agent in the pharmaceutically acceptable formulation is mannitol at a concentration of about 15 mg/mL to about 55 mg/mL.

In some aspects, the osmotic agent in the pharmaceutically acceptable formulation is mannitol at a concentration of about 20 mg/mL to about 50 mg/mL.

In some aspects, the osmotic agent in the pharmaceutically acceptable formulation is mannitol at a concentration of about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL.

In some aspects, the osmotic agent in the pharmaceutically acceptable formulation is mannitol at a concentration of about 25 mg/mL to about 45 mg/mL.

In some aspects, the osmotic agent in the pharmaceutically acceptable formulation is mannitol at a concentration of about 30 mg/mL to about 40 mg/mL.

In some aspects, the osmotic agent in the pharmaceutically acceptable formulation is sucrose.

In some aspects, the osmotic agent in the pharmaceutically acceptable formulation is sucrose at a concentration of about 0.5 mg/mL to about 25 mg/mL.

In some aspects, the osmotic agent in the pharmaceutically acceptable formulation is sucrose at a concentration of about 2 mg/mL to about 20 mg/mL.

In some aspects, the osmotic agent in the pharmaceutically acceptable formulation is sucrose at a concentration of about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation is sucrose at a concentration of about 4 mg/mL to about 18 mg/mL.

In some aspects, the osmotic agent in the pharmaceutically acceptable formulation is sucrose at a concentration of about 6 mg/mL to about 16 mg/mL.

In some aspects, the pharmaceutically acceptable formulation further comprises an antioxidant.

In some aspects, the pharmaceutically acceptable formulation comprises an antioxidant selected from the group consisting of methionine, monothioglycerol, sodium bisulfite, sodium metabisulfite, sodium thiosulfate, sodium citrate, and thiourea, or combinations thereof.

In some aspects, the antioxidant in the pharmaceutically acceptable formulation is methionine. In some aspects, the concentration of methionine in the pharmaceutically acceptable formulation is about 1.5 mg/mL to about 5 mg/mL. In some aspects, the concentration of methionine in the pharmaceutically acceptable formulation is about 1.5 mg/mL, about 2 mg/mL, about 2.5 mg/mL, about 3 mg/mL, about 3.5 mg/mL, about 4 mg/mL, about 4.5 mg/mL, or about 5 mg/mL.

In some aspects, the antioxidant in the pharmaceutically acceptable formulation is monothioglycerol. In some aspects, the concentration of monothioglycerol in the pharmaceutically acceptable formulation is about 2 mg/mL to about 3 mg/mL. In some aspects, the concentration of monothioglycerol in the pharmaceutically acceptable formulation is about 2 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, or about 3 mg/mL.

In some aspects, the antioxidant in the pharmaceutically acceptable formulation is sodium bisulfite.

In some aspects, the antioxidant in the pharmaceutically acceptable formulation is sodium metabisulfite.

In some aspects, the concentration of sodium metabisulfite in the pharmaceutically acceptable formulation is about 0.1 mg/mL to about 5.0 mg/mL.

In some aspects, the concentration of sodium metabisulfite in the pharmaceutically acceptable formulation is about 0.2 mg/mL to about 4.0 mg/mL.

In some aspects, the concentration of sodium metabisulfite in the pharmaceutically acceptable formulation is about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium metabisulfite in the pharmaceutically acceptable formulation is about 0.5 mg/mL to about 3.5 mg/mL.

In some aspects, the concentration of sodium metabisulfite in the pharmaceutically acceptable formulation is about 1.0 mg/mL to about 3.0 mg/mL.

In some aspects, the antioxidant in the pharmaceutically acceptable formulation is sodium thiosulfate.

In some aspects, the antioxidant in the pharmaceutically acceptable formulation is sodium citrate.

In some aspects, the concentration of sodium citrate in the pharmaceutically acceptable formulation is about 0.05 mg/mL to about 5.0 mg/mL.

In some aspects, the concentration of sodium citrate in the pharmaceutically acceptable formulation is about 0.1 mg/mL to about 4.0 mg/mL.

In some aspects, the concentration of sodium citrate in the pharmaceutically acceptable formulation is about 0.1 mg/mL, about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium citrate in the pharmaceutically acceptable formulation is about 0.5 mg/mL to about 3.5 mg/mL.

In some aspects, the concentration of sodium citrate in the pharmaceutically acceptable formulation is about 1.0 mg/mL to about 3.0 mg/mL.

In some aspects, the antioxidant in the pharmaceutically acceptable formulation is thiourea.

In some aspects, the pharmaceutically acceptable formulation further comprises a cyclodextrin.

In some aspects, the pharmaceutically acceptable formulation comprises a cyclodextrin selected from the group consisting of 2-hydroxypropyl-β-cyclodextrin, O-methyl-β-cyclodextrin, and γ-cyclodextrin, or combinations thereof.

In some aspects, the cyclodextrin in the pharmaceutically acceptable formulation is 2-hydroxypropyl-β-cyclodextrin.

In some aspects, the cyclodextrin in the pharmaceutically acceptable formulation is O-methyl-β-cyclodextrin.

In some aspects, the cyclodextrin in the pharmaceutically acceptable formulation is γ-cyclodextrin.

In some aspects, an injector comprises the pharmaceutically acceptable formulation.

In some aspects, the pharmaceutically acceptable formulation can be dispensed from an injector.

In some aspects, the pharmaceutically acceptable formulation can be dispensed from an injector, where the injector is an autoinjector.

In some aspects, the pharmaceutically acceptable formulation can be dispensed from an injector, where the injector is a pen injector.

In some aspects, the pharmaceutically acceptable formulation can be dispensed from an injector, where the injector is a needle-less injector.

In some aspects, an injector can be adjusted to dispense about 0.05 mL to about 0.5 mL of the pharmaceutically acceptable formulation.

In some aspects, an injector can be adjusted to dispense about 0.1 mL to about 0.3 mL of the pharmaceutically acceptable formulation.

In some aspects, an injector can be adjusted to dispense about 0.10 mL, about 0.15 mL, about 0.20 mL, about 0.25 mL, or about 0.3 mL of the pharmaceutically acceptable formulation.

In some aspects, an injector can be adjusted to dispense about 0.18 mL to about 0.27 mL of the pharmaceutically acceptable formulation.

In some aspects, an injector can be adjusted to dispense about 0.15 mL to about 0.25 mL of the pharmaceutically acceptable formulation.

In some aspects, the pharmaceutically acceptable formulation is contained within a sterilized cartridge with a capacity of about 3.0 mL, which can be operably attached to an injector to dispense a quantity of the pharmaceutically acceptable formulation.

In some aspects, a pre-filled injector comprises a cartridge comprising the pharmaceutically acceptable formulation.

In some aspects, the injector can dispense about 10 injections per cartridge.

In some aspects, the preparation of the pharmaceutically acceptable formulation comprises adjustment of the pharmaceutically acceptable formulation to a target pH of about 2.5 and about 4.5 with methanesulfonic acid, carbon dioxide, and/or sodium hydroxide.

In some aspects, the pharmaceutically acceptable formulation is stable.

In some aspects, the pharmaceutically acceptable formulation is stable as determined by a substantially unchanged amount of DHE following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the pharmaceutically acceptable formulation is stable as determined by a substantially unchanged amount of one or more impurities following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the one or more impurities comprise Impurity A. In some aspects, the one or more impurities comprise Impurity B. In some aspects, the one or more impurities comprise Impurity C. In some aspects, the one or more impurities comprise Impurity D. In some aspects, the one or more impurities comprise Impurity E. In some aspects, the one or more impurities comprise Impurity A-E.

In some aspects, the pharmaceutically acceptable formulation is substantially free of one or more of impurities selected from the group consisting of Impurity A, Impurity B, Impurity C, Impurity D, and Impurity E.

### Method of Treatment

The present disclosure also provides a method of treating head pain in a patient, the method comprising administration to a patient in need thereof a therapeutically effective amount of a pharmaceutically acceptable formulation, the formulation comprising DHE mesylate at a concentration of about 3 mg/mL to about 6 mg/mL and carbon dioxide at a concentration sufficient to retard oxidative degradation of DHE mesylate.

In some aspects, the head pain to be treated is a cluster headache attack.

In some aspects, the head pain to be treated is a migraine.

In some aspects, the pharmaceutically acceptable formulation of the method of treatment further comprises caffeine.

In some aspects, the caffeine in the pharmaceutically acceptable formulation of the method of treatment is at a concentration of about 1 mg/mL to about 20 mg/mL.

In some aspects, the caffeine in the pharmaceutically acceptable formulation of the method of treatment is at a concentration of about 5 mg/mL to about 15 mg/mL.

In some aspects, the caffeine in the pharmaceutically acceptable formulation of the method of treatment is at a concentration of about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, or about 15 mg/mL.

In some aspects, the caffeine in the pharmaceutically acceptable formulation of the method of treatment is at a concentration of about 6 mg/mL to about 14 mg/mL.

In some aspects, the caffeine in the pharmaceutically acceptable formulation of the method of treatment is at a concentration of about 7 mg/mL to about 13 mg/mL.

In some aspects, the pharmaceutically acceptable formulation of the method of treatment further comprises an osmotic agent.

In some aspects, the pharmaceutically acceptable formulation of the method of treatment comprises an osmotic agent selected from the group consisting of: dextrose, glycerin, mannitol, and sucrose, or combinations thereof.

In some aspects, the osmotic agent in the pharmaceutically acceptable formulation of the method of treatment is dextrose.

In some aspects, the osmotic agent in the pharmaceutically acceptable formulation of the method of treatment is dextrose at a concentration of about 0.1 mg/mL to about 25 mg/mL.

In some aspects, the osmotic agent in the pharmaceutically acceptable formulation of the method of treatment is dextrose at a concentration of about 1 mg/mL to about 20 mg/mL.

In some aspects, the osmotic agent in the pharmaceutically acceptable formulation is dextrose at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation of the method of treatment is dextrose at a concentration of about 3 mg/mL to about 18 mg/mL.

In some aspects, the osmotic agent in the pharmaceutically acceptable formulation of the method of treatment is dextrose at a concentration of about 5 mg/mL to about 16 mg/mL.

In some aspects, the osmotic agent in the pharmaceutically acceptable formulation of the method of treatment is glycerin.

In some aspects, the osmotic agent in the pharmaceutically acceptable formulation of the method of treatment is glycerin at a concentration of about 0.1 mg/mL to about 25 mg/mL.

In some aspects, the osmotic agent in the pharmaceutically acceptable formulation of the method of treatment is glycerin at a concentration of about 1 mg/mL to about 20 mg/mL.

In some aspects, the osmotic agent in the pharmaceutically acceptable formulation is glycerin at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation of the method of treatment is glycerin at a concentration of about 3 mg/mL to about 18 mg/mL.

In some aspects, the osmotic agent in the pharmaceutically acceptable formulation of the method of treatment is glycerin at a concentration of about 5 mg/mL to about 16 mg/mL.

In some aspects, the osmotic agent in the pharmaceutically acceptable formulation of the method of treatment is mannitol.

In some aspects, the osmotic agent in the pharmaceutically acceptable formulation of the method of treatment is mannitol at a concentration of about 15 mg/mL to about 55 mg/mL.

In some aspects, the osmotic agent in the pharmaceutically acceptable formulation of the method of treatment is mannitol at a concentration of about 20 mg/mL to about 50 mg/mL.

In some aspects, the osmotic agent in the pharmaceutically acceptable formulation is mannitol at a concentration of about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL.

In some aspects, the osmotic agent in the pharmaceutically acceptable formulation of the method of treatment is mannitol at a concentration of about 25 mg/mL to about 45 mg/mL.

In some aspects, the osmotic agent in the pharmaceutically acceptable formulation of the method of treatment is mannitol at a concentration of about 30 mg/mL to about 40 mg/mL.

In some aspects, the osmotic agent in the pharmaceutically acceptable formulation of the method of treatment is sucrose.

In some aspects, the osmotic agent in the pharmaceutically acceptable formulation of the method of treatment is sucrose at a concentration of about 0.5 mg/mL to about 25 mg/mL.

In some aspects, the osmotic agent in the pharmaceutically acceptable formulation of the method of treatment is sucrose at a concentration of about 2 mg/mL to about 20 mg/mL.

In some aspects, the osmotic agent in the pharmaceutically acceptable formulation is sucrose at a concentration of about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent in the pharmaceutically acceptable formulation of the method of treatment is sucrose at a concentration of about 4 mg/mL to about 18 mg/mL.

In some aspects, the osmotic agent in the pharmaceutically acceptable formulation of the method of treatment is sucrose at a concentration of about 6 mg/mL to about 16 mg/mL.

In some aspects, the pharmaceutically acceptable formulation of the method of treatment further comprises an antioxidant.

In some aspects, the pharmaceutically acceptable formulation of the method of treatment comprises an antioxidant selected from the group consisting of: methionine, monothioglycerol, sodium bisulfite, sodium metabisulfite, sodium thiosulfate, sodium citrate, and thiourea, or combinations thereof.

In some aspects, the antioxidant in the pharmaceutically acceptable formulation of the method of treatment is methionine.

In some aspects, the concentration of methionine in the pharmaceutically acceptable formulation is about 1.5 mg/mL to about 5 mg/mL. In some aspects, the concentration of methionine is about 1.5 mg/mL, about 2 mg/mL, about 2.5 mg/mL, about 3 mg/mL, about 3.5 mg/mL, about 4 mg/mL, about 4.5 mg/mL, or about 5 mg/mL.

In some aspects, the antioxidant in the pharmaceutically acceptable formulation of the method of treatment is monothioglycerol.

In some aspects, the concentration of monothioglycerol in the pharmaceutically acceptable formulation is about 2 mg/mL to about 3 mg/mL. In some aspects, the concentration of monothioglycerol in the pharmaceutically acceptable formulation is about 2 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, or about 3 mg/mL.

In some aspects, the antioxidant in the pharmaceutically acceptable formulation of the method of treatment is sodium bisulfite.

In some aspects, the antioxidant in the pharmaceutically acceptable formulation of the method of treatment is sodium metabisulfite.

In some aspects, the concentration of sodium metabisulfite in the pharmaceutically acceptable formulation is about 0.1 mg/mL to about 5.0 mg/mL.

In some aspects, the concentration of sodium metabisulfite in the pharmaceutically acceptable formulation is about 0.2 mg/mL to about 4.0 mg/mL.

In some aspects, the concentration of sodium metabisulfite in the pharmaceutically acceptable formulation is about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium metabisulfite in the pharmaceutically acceptable formulation is about 0.5 mg/mL to about 3.5 mg/mL.

In some aspects, the concentration of sodium metabisulfite in the pharmaceutically acceptable formulation is about 1.0 mg/mL to about 3.0 mg/mL.

In some aspects, the antioxidant in the pharmaceutically acceptable formulation of the method of treatment is sodium thiosulfate.

In some aspects, the antioxidant in the pharmaceutically acceptable formulation of the method of treatment is sodium citrate.

In some aspects, the concentration of sodium citrate in the pharmaceutically acceptable formulation is about 0.05 mg/mL to about 5.0 mg/mL.

In some aspects, the concentration of sodium citrate in the pharmaceutically acceptable formulation is about 0.1 mg/mL to about 4.0 mg/mL.

In some aspects, the concentration of sodium citrate in the pharmaceutically acceptable formulation is about 0.1 mg/mL, about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium citrate in the pharmaceutically acceptable formulation is about 0.5 mg/mL to about 3.5 mg/mL.

In some aspects, the concentration of sodium citrate in the pharmaceutically acceptable formulation is about 1.0 mg/mL to about 3.0 mg/mL.

In some aspects, the antioxidant in the pharmaceutically acceptable formulation of the method of treatment is thiourea.

In some aspects, the pharmaceutically acceptable formulation of the method of treatment further comprises a cyclodextrin.

In some aspects, the cyclodextrin in the pharmaceutically acceptable formulation of the method of treatment is 2-hydroxypropyl-β-cyclodextrin.

In some aspects, the cyclodextrin in the pharmaceutically acceptable formulation of the method of treatment is O-methyl-β-cyclodextrin.

In some aspects, the cyclodextrin in the pharmaceutically acceptable formulation of the method of treatment is γ-cyclodextrin.

In some aspects, the pharmaceutically acceptable formulation further comprises a preservative.

In some aspects, the preservative is an isomer of cresol selected from the group consisting of o-cresol, m-cresol, and p-cresol, or combinations thereof. In some aspects, the preservative is o-cresol. In some aspects, the preservative is p-cresol.

In some aspects, the preservative is m-cresol. In some aspects, m-cresol is present at a concentration of about 1 mg/mL to about 5 mg/mL. In some aspects, m-cresol is present at a concentration of about 1 mg/mL to about 2.5 mg/mL. In some aspects, m-cresol is present at a concentration of about 1.5 mg/mL.

In some aspects, the preservative is benzyl alcohol. In some aspects, benzyl alcohol is present at a concentration of about 5 mg/mL to about 100 mg/mL. In some aspects, benzyl alcohol is present at a concentration of about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 18 mg/mL, about 19 mg/mL, about 20 mg/mL, about 25 mg/mL, about 30 mg/mL, about 35 mg/mL, about 40 mg/mL, about 50 mg/mL, about 60 mg/mL, about 70 mg/mL, about 80 mg/mL, about 90 mg/mL, or about 100 mg/mL. In some aspects, benzyl alcohol is present at a concentration of about 10 mg/mL.

In some aspects, the pharmaceutically acceptable formulation is stable.

In some aspects, the pharmaceutically acceptable formulation is stable as determined by a substantially unchanged amount of DHE following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the pharmaceutically acceptable formulation is stable as determined by a substantially unchanged amount of one or more impurities following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the one or more impurities comprise Impurity A. In some aspects, the one or more impurities comprise Impurity B. In some aspects, the one or more impurities comprise Impurity C. In some aspects, the one or more impurities comprise Impurity D. In some aspects, the one or more impurities comprise Impurity E.

In some aspects, the pharmaceutically acceptable formulation is substantially free of one or more of impurities selected from the group consisting of Impurity A, Impurity B, Impurity C, Impurity D, and Impurity E.

In some aspects, the method of treatment further comprises subcutaneous administration of the pharmaceutically acceptable formulation to the patient.

In some aspects, the method of treatment further comprises intramuscular administration of the pharmaceutically acceptable formulation to the patient.

In some aspects, the method of treatment further comprises intravenous administration of the pharmaceutically acceptable formulation to the patient.

In some aspects, the method of treatment comprises administration of the pharmaceutically acceptable formulation to the patient from an injector pre-loaded with the pharmaceutically acceptable formulation.

In some aspects, the method of treatment further comprises administration of about 0.05 mL to about 0.5 mL of the pharmaceutically acceptable formulation.

In some aspects, the method of treatment further comprises administration of about 0.1 mL to about 0.3 mL of the pharmaceutically acceptable formulation.

In some aspects, the method of treatment comprises administration of the pharmaceutically acceptable formulation of about 0.10 mL, about 0.15 mL, about 0.20 mL, about 0.25 mL, or about 0.30 mL.

In some aspects, the method of treatment further comprises administration of about 0.13 to about 0.27 mL of the pharmaceutically acceptable formulation.

In some aspects, the method of treatment further comprises administration of about 0.15 to about 0.25 mL of the pharmaceutically acceptable formulation.

In some aspects, the pharmaceutically acceptable formulation of the method of treatment is administered at a quantity sufficient to eliminate or reduce head pain.

In some aspects, the method of treatment reduces the severity, duration, or occurrence of headaches or migraines experienced by the patient.

In some aspects, the method of treatment is performed prior to the onset of head pain.

In some aspects, the method of treatment is performed prior to the most severe symptoms of an episode of head pain.

In some aspects, the method of treatment reduces the severity of an oncoming episode of head pain.

In some aspects, the method of treatment prevents the onset of head pain.

In some aspects, the pharmaceutically acceptable formulation of the method of treatment is sparged with carbon dioxide to achieve a chosen concentration of carbon dioxide.

In some aspects, the method of treatment further comprises subcutaneous administration of the pharmaceutically acceptable formulation to the patient.

In some aspects, the method of treatment further comprises intramuscular administration of the pharmaceutically acceptable formulation to the patient.

In some aspects, the method of treatment further comprises intravenous administration of the pharmaceutically acceptable formulation to the patient.

In some aspects, the method of treatment further comprises administration of the pharmaceutically acceptable formulation to a patient from an injector, which comprises the pharmaceutically acceptable formulation.

In some aspects, an injector can dispense about 10 injections per cartridge.

In some aspects, the method of treatment includes multiple injections within the same minute, hour, day, week, or month.

In some aspects, a patient suitable for the method of treatment is a nonhuman animal. In some aspects, a patient suitable for the method of treatment is a mammal. In some aspects, a patient suitable for the method of treatment is a non-primate, e.g., rabbit, cow, pig, horse, cat, dog, rat, or a primate, such as a Cynomolgous monkey. In some aspects, a patient suitable for the method of treatment is a human. In some aspects, a patient suitable for the method of treatment is a human male. In some aspects, a patient suitable for the method of treatment is a human male of age 50 or older. In some aspects, a patient suitable for the method of treatment is a human female. In some aspects, a patient suitable for the method of treatment is a human female of age 50 or older. In some aspects, a patient suitable for the method of treatment is a pre-menopause human female. In some aspects, a patient suitable for the method of treatment is a perimenopause human female. In some aspects, a patient suitable for the method of treatment is a menopausal human female. In some aspects, a patient suitable for the method of treatment is a post- menopause human female. In some aspects, a patient suitable for the method of treatment is a pregnant human female. In some aspects, a patient suitable for the method of treatment is a human who suffers from migraines with aura. In some aspects, a patient suitable for the method of treatment is a human who suffers from migraines without aura. In some aspects, a patient suitable for the method of treatment is a human who suffers from cluster headaches. In some aspects, a patient suitable for the method of treatment is a human with a diagnosed disorder, which presents at least in part with head pain.

In some aspects, a patient suitable for the method of treatment is a child of about age 5 or younger.

In some aspects, a patient suitable for the method of treatment is a child of about age 6 to about age 12.

In some aspects, a patient suitable for the method of treatment is an adolescent of about age 13 to about age 17.

In some aspects, a patient suitable for the method of treatment is an adult of about age 18 or older. In some aspects, the amount of DHE mesylate administered subcutaneously to a patient of age 18 or older is about 1 mg per injection.

In some aspects, the amount of DHE mesylate administered to a patient of age 18 or older does not exceed intramuscular injection of about 3 mg per 24 hour period, or intravenous injection of about 2 mg per 24 hour period, or any route of administration of about 6 mg per week.

In some aspects, patients of ages 6-12 can receive about 1 mg intravenous administration of DHE mesylate over the course of about 3 minutes every about 8 hours as needed.

In some aspects, patients of ages 13-17 can receive about 1 mg intravenous administration of DHE mesylate over the course of about 3 minutes every about 8 hours as needed.

In some aspects, the dose administered is decreased by about 0.5 mg every eight hours for patients who weigh less than about 25 kg or are under age 10.

In some aspects, patients of ages 6-12, or patients of ages 13-17, or patients who weigh less than about 25 kg, or patients under age 10 receive no more than about 1 mg intravenous DHE per hour.

In some aspects, patients of age 18 or older, including the elderly, receive no more than about 1 mg DHE mesylate per dose, not to exceed intramuscular administration of about 3 mg per 24 hour period. In some aspects, patients of age 18 or older, including the elderly, receive no more than about 1 mg DHE per dose, not to exceed intravenous administration of about 2 mg per 24 hour period. In some aspects, patients of age 18 or older, including the elderly, receive no more than about 6 mg DHE mesylate per week by intramuscular or intravenous administration.

In some aspects, patients of ages 6-12 or patients of ages 13-17 receive no more than about 0.5 mg DHE mesylate per intramuscular dose, not to exceed 2 doses per 24 hour period, and not to exceed about 1 mg per week. In some aspects, patients of ages 6-12 or patients of ages 13-17 receive no more than about 0.25 mg DHE mesylate per intravenous dose, not to exceed 2 doses per 24 hour period, and not to exceed about 1 mg per week.

In further aspects, DHE formulations may be administered via a parenteral route. As used herein, the term “parenteral” includes routes that bypass the alimentary tract. Specifically, the pharmaceutical compositions disclosed herein can be administered for example, but not limited to intravenously, intradermally, intramuscularly, intraarterially, intrathecally, subcutaneous, or intraperitoneally.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In some aspects, the pharmaceutically acceptable formulation is administered at a quantity sufficient to eliminate or reduce head pain. In some aspects, the method of treatment reduces the severity, duration, or occurrence of headaches or migraines experienced by the patient.

In some aspects, the preparation of the pharmaceutically acceptable formulation comprises adjustment of the pharmaceutically acceptable formulation to a target pH of about 2.5 and about 4.5 with methanesulfonic acid, carbon dioxide, and/or sodium hydroxide.

### Pre-Filled Injector Method of Manufacture

The present disclosure further provides a method of manufacturing a pre-filled injector comprising a pharmaceutically acceptable formulation comprising about 3 mg/mL to about 6 mg/mL DHE mesylate, about 3 mg/mL to about 10 mg/mL of a pharmaceutically acceptable alcohol, about 5 mg/mL to about 15 mg/mL caffeine, about 1 mg/mL to about 3 mg/mL an isomer of cresol (e.g., o-cresol, m-cresol, or p-cresol), and an osmotic agent; sparging the pharmaceutically acceptable formulation with carbon dioxide at a concentration sufficient to retard oxidative degradation of the DHE mesylate; loading a sterile cartridge with the pharmaceutically acceptable formulation; and attaching the sterile cartridge comprising the pharmaceutically acceptable formulation operably to an injector.

In some aspects, the pharmaceutically acceptable alcohol is selected from the group consisting of: propylene glycol, ethanol, and a polyethylene glycol, or combinations thereof.

In some aspects, the pharmaceutically acceptable alcohol is ethanol.

In some aspects, the cartridge has a capacity of about 3.0 mL.

In some aspects, the osmotic agent is dextrose.

In some aspects, the osmotic agent is dextrose at a concentration of about 0.1 mg/mL to about 25 mg/mL.

In some aspects, the osmotic agent is dextrose at a concentration of about 1 mg/mL to about 20 mg/mL.

In some aspects, the osmotic agent is dextrose at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent is dextrose at a concentration of about 3 mg/mL to about 18 mg/mL.

In some aspects, the osmotic agent is dextrose at a concentration of about 5 mg/mL to about 16 mg/mL.

In some aspects, the osmotic agent is glycerin.

In some aspects, the osmotic agent is glycerin at a concentration of about 0.1 mg/mL to about 25 mg/mL.

In some aspects, the osmotic agent is glycerin at a concentration of about 1 mg/mL to about 20 mg/mL.

In some aspects, the osmotic agent is glycerin at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent is glycerin at a concentration of about 3 mg/mL to about 18 mg/mL.

In some aspects, the osmotic agent is glycerin at a concentration of about 5 mg/mL to about 16 mg/mL.

In some aspects, the osmotic agent is mannitol.

In some aspects, the osmotic agent is mannitol at a concentration of about 15 mg/mL to about 55 mg/mL.

In some aspects, the osmotic agent is mannitol at a concentration of about 20 mg/mL to about 50 mg/mL.

In some aspects, the osmotic agent is mannitol at a concentration of about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL.

In some aspects, the osmotic agent is mannitol at a concentration of about 25 mg/mL to about 45 mg/mL.

In some aspects, the osmotic agent is mannitol at a concentration of about 30 mg/mL to about 40 mg/mL.

In some aspects, the osmotic agent is sucrose.

In some aspects, the osmotic agent is sucrose at a concentration of about 0.2 mg/mL to about 25 mg/mL.

In some aspects, the osmotic agent is sucrose at a concentration of about 2 mg/mL to about 20 mg/mL.

In some aspects, the osmotic agent is sucrose at a concentration of about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

In some aspects, the osmotic agent is sucrose at a concentration of about 4 mg/mL to about 18 mg/mL.

In some aspects, the osmotic agent is sucrose at a concentration of about 6 mg/mL to about 16 mg/mL.

In some aspects, the pharmaceutically acceptable formulation further comprises a cyclodextrin.

In some aspects, the pharmaceutically acceptable formulation comprises a cyclodextrin selected from the group consisting of: 2-hydroxypropyl-β-cyclodextrin, O-methyl-β-cyclodextrin, and γ-cyclodextrin, or combinations thereof.

In some aspects, the cyclodextrin is 2-hydroxypropyl-β-cyclodextrin.

In some aspects, the cyclodextrin is O-methyl-β-cyclodextrin.

In some aspects, the cyclodextrin is γ-cyclodextrin.

In some aspects, the pharmaceutically acceptable formulation further comprises an antioxidant.

In some aspects, the pharmaceutically acceptable formulation comprises an antioxidant selected from the group consisting of: methionine, monothioglycerol, sodium bisulfite, sodium metabisulfite, sodium thiosulfate, sodium citrate, and thiourea, or combinations thereof.

In some aspects, the antioxidant is methionine. In some aspects, the concentration of methionine is about 1.5 mg/mL to about 5 mg/mL. In some aspects, the concentration of methionine is about 1.5 mg/mL, about 2 mg/mL, about 2.5 mg/mL, about 3 mg/mL, about 3.5 mg/mL, about 4 mg/mL, about 4.5 mg/mL, or about 5 mg/mL.

In some aspects, the antioxidant is monothioglycerol. In some aspects, the concentration of monothioglycerol is about 2 mg/mL to about 3 mg/mL. In some aspects, the concentration of monothioglycerol is about 2 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, or about 3 mg/mL.

In some aspects, the antioxidant is sodium bisulfite.

In some aspects, the antioxidant is sodium metabisulfite.

In some aspects, the concentration of sodium metabisulfite is about 0.1 mg/mL to about 5.0 mg/mL.

In some aspects, the concentration of sodium metabisulfite is about 0.2 mg/mL to about 4.0 mg/mL.

In some aspects, the concentration of sodium metabisulfite is about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium metabisulfite is about 0.5 mg/mL to about 3.5 mg/mL.

In some aspects, the concentration of sodium metabisulfite is about 1.0 mg/mL to about 3.0 mg/mL.

In some aspects, the antioxidant is sodium thiosulfate.

In some aspects, the antioxidant is sodium citrate.

In some aspects, the concentration of sodium citrate is about 0.05 mg/mL to about 5.0 mg/mL.

In some aspects, the concentration of sodium citrate is about 0.1 mg/mL to about 4.0 mg/mL.

In some aspects, the concentration of sodium citrate is about 0.1 mg/mL, about 0.2 mg/mL, about 0.4 mg/mL, about 0.6 mg/mL, about 0.8 mg/mL, about 1.0 mg/mL, about 1.2 mg/mL, about 1.4 mg/mL, about 1.6 mg/mL, about 1.8 mg/mL, about 2.0 mg/mL, about 2.2 mg/mL, about 2.4 mg/mL, about 2.6 mg/mL, about 2.8 mg/mL, about 3.0 mg/mL, about 3.2 mg/mL, about 3.4 mg/mL, about 3.6 mg/mL, about 3.8 mg/mL, or about 4.0 mg/mL.

In some aspects, the concentration of sodium citrate is about 0.5 mg/mL to about 3.5 mg/mL.

In some aspects, the concentration of sodium citrate is about 1.0 mg/mL to about 3.0 mg/mL.

In some aspects, the antioxidant is thiourea.

In some aspects, the pharmaceutically acceptable formulation is stable.

In some aspects, the pharmaceutically acceptable formulation is stable as determined by a substantially unchanged amount of DHE following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the pharmaceutically acceptable formulation is stable as determined by a substantially unchanged amount of one or more impurities following storage in a sealed vial at 40° C. for a period of about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, or about 36 months.

In some aspects, the one or more impurities comprise Impurity A. In some aspects, the one or more impurities comprise Impurity B. In some aspects, the one or more impurities comprise Impurity C. In some aspects, the one or more impurities comprise Impurity D. In some aspects, the one or more impurities comprise Impurity E. In some aspects, the one or more impurities comprise Impurity A-E.

In some aspects, the pharmaceutically acceptable formulation is substantially free of one or more of impurities selected from the group consisting of Impurity A, Impurity B, Impurity C, Impurity D, and Impurity E.

In some aspects, the method of manufacture of the injector comprises inclusion in the injector of an adjustable input mechanism, such that the volume to be dispensed from the injector is selectable.

In some aspects, the method of manufacture of the injector comprises inclusion in the injector of an adjustable input mechanism, such that the volume to be dispensed from the injector is adjustable to a volume of about 0.05 mL to about 0.5 mL.

In some aspects, the method of manufacture of the injector comprises inclusion in the injector of an adjustable input mechanism, such that the volume to be dispensed from the injector is adjustable to a volume of about 0.1 mL to about 0.3 mL.

In some aspects, the method of manufacture of the injector comprises inclusion in the injector of an adjustable input mechanism, such that the volume to be dispensed from the injector is adjustable to a volume of about 0.10 mL, about 0.15 mL, about 0.20 mL, about 0.25 mL, or about 0.30 mL.

In some aspects, the method of manufacture of the injector comprises inclusion in the injector of an adjustable input mechanism, such that the volume to be dispensed from the injector is adjustable to a volume of about 0.18 mL to about 0.27 mL.

In some aspects, the method of manufacture of the injector comprises inclusion in the injector of an adjustable input mechanism, such that the volume to be dispensed from the injector is adjustable to a volume of about 0.15 mL to about 0.25 mL.

In some aspects, the pre-filled injector can dispense about 10 injections per cartridge.

In some aspects, the pre-filled injector can dispense about 1, about 2, about 3, about 4, about 5, about 6, about 7, about 8, about 9, about 10, about 11, about 12, about 13, about 14, or about 15 injections per cartridge.

In some aspects, the preparation of the pharmaceutically acceptable formulation comprises adjustment of the pharmaceutically acceptable formulation to a target pH of about 2.5 and about 4.5 with methanesulfonic acid, carbon dioxide, and/or sodium hydroxide.

In some aspects, an injector herein may be understood as an injector that, upon actuation (e.g., pressing of a button), a syringe needle is automatically inserted and the subject DHE formulation is delivered at a selected dose.

In some aspects, the injector is a spring loaded device.

In some aspects, the injector is a needle-less device.

In some aspects, the injector comprises a first disposable needle which can be detached from the injector by a user and replaced with a second disposable needle.

In some aspects, once the injection is complete the injectors may provide an indication to the user to confirm that a particular dose has been delivered. In such context, injectors herein may include gas jet injectors which contain a cylinder of pressurized gas that propels a jet of the liquid dose through the skin without the use of a needle.

In some aspects, the injector contents herein may include other optional components, in relatively small amounts, such as 1-50 mg of other excipients (inert ingredients with respect to drug activity).

In some aspects, excipients can be incorporated to improve the flow of the DHE formulation, as well as its stability, when used in an injector.

### Conditions to be Treated

The present disclosure relates to a method of reducing the severity of, preventing, or eliminating head pain caused by: primary headaches, including tension headaches, migraine headaches, and cluster headaches; secondary headaches including traumatic headaches such as post-concussion headaches, headaches associated with substance abuse, headaches associated with medicine or other chemical overdose, headaches caused by dehydration, headaches stemming from dental or brain infections, and headaches associated with underlying head or neck damage; and cranial neuralgias, including trigeminal neuralgia. Types of headaches which can be treated include: primary tension headaches that are episodic, primary tension headaches that are chronic, primary muscle contraction headaches, primary migraine headaches with aura, primary migraine headaches without aura, primary cluster headache, primary paroxysmal hemicranias, primary cough headache, primary stabbing headache, primary headache associated with sexual intercourse, primary thunderclap headache, hypnic headache, hemicrania continua, new daily-persistent headache, headache from exertion, trigeminal neuralgia and other cranial nerve inflammation, and secondary headaches due to trauma, disorders, infection, substance abuse or withdrawal, or structural problems with the bones of the face, teeth, eyes, ears, nose, sinuses, or other structures.

### Combination Therapy

In some aspects, a pharmaceutically acceptable formulation comprising DHE mesylate can be administered in combination with one or more additional therapeutic agents, in a single dosage form or as separate dosage forms.

In some aspects, when administered as a separate dosage form, DHE mesylate may be administered prior to, concurrently as, or following administration of one or more additional therapeutic agents. In some embodiments, when administered as a separate dosage form, one or more doses of one or more additional therapeutic agents may be administered prior to the DHE mesylate.

As used herein, the administration in “combination” of DHE mesylate, and one or more additional therapeutic agents refers not only to simultaneous or sequential administration of the agents, but also to the administration of the agents during a single treatment cycle, as understood by one skilled in the art.

In some aspects, the one or more additional therapeutic agents can be heparin or a local anesthetic.

In some aspects, a pharmaceutically acceptable formulation comprising DHE mesylate can further comprise an anticoagulant, which can be heparin or a pharmaceutically acceptable salt thereof.

In some aspects, a pharmaceutically acceptable formulation comprising DHE mesylate can further comprise a local anesthetic, which can be lidocaine or a pharmaceutically acceptable salt thereof.

### Pharmacokinetics

In some aspects, DHE is about 90% to about 93% bound to plasma proteins at some time following administration of a pharmaceutically acceptable formulation of DHE mesylate.

In some aspects, the volume of distribution of DHE is about 800 liters.

In some aspects, DHE is metabolized in the liver, producing metabolites 8′-β-hydroxydihydroergotamine and dihydrolysergic acid amide. In some aspects these metabolites are further metabolized to a carboxylic acid derivative of 8′-β-hydroxydihydroergotamine, and dihydrolysergic acid, respectively.

In some aspects, about 10% of a dose of DHE is excreted renally. In some aspects, about 90% of a dose of DHE is excreted through the biliary-fecal route.

In some aspects, the total body clearance of DHE is about 1.5 L/min.

In some aspects, about 6-7% of DHE administered intramuscularly is excreted in the urine.

In some aspects, the rate of renal clearance of DHE administered by any route is about 0.1 L/min.

In some aspects, pain relief is observed within about 5 minutes of intravenous treatment with a pharmaceutically acceptable formulation comprising DHE mesylate.

In some aspects, pain relief is observed within about 15 to about 30 minutes following intramuscular treatment with a pharmaceutically acceptable formulation comprising DHE mesylate. In some aspects, the intramuscular delivery results in pain relief for about 3 to about 4 hours.

In some aspects, the peak plasma concentration of DHE is reached within about 30 to about 60 minutes following subcutaneous administration of a pharmaceutically acceptable formulation of DHE mesylate.

In some aspects, the decline of plasma DHE after intramuscular or intravenous administration of the pharmaceutically acceptable formulation is multi-exponential with a terminal half-life of about 9 hours.

In some aspects, the method of treatment provides about 9 hours of relief from head pain. In some aspects, the method of treatment provides pain relief for about 0.5 hours, about 1.0 hour, about 1.5 hours, about 2.0 hours, about 2.5 hours, about 3.0 hours, about 3.5 hours, about 4.0 hours, about 4.5 hours, about 5.0 hours, about 5.5 hours, about 6.0 hours, about 6.5 hours, about 7.0 hours, about 7.5 hours, about 8.0 hours, about 8.5 hours, about 9.0 hours, about 9.5 hours, about 10.0 hours, about 10.5 hours, about 11.0 hours, about 11.5 hours, about 12.0 hours, about 12.5 hours, about 13.0 hours, about 13.5 hours, about 14.0 hours, about 14.5 hours, about 15.0 hours, about 15.5 hours, about 16.0 hours, about 16.5 hours, about 17.0 hours, about 17.5 hours, about 18.0 hours, about 18.5 hours, about 19.0 hours, about 19.5 hours, about 20.0 hours, about 20.5 hours, about 21.0 hours, about 21.5 hours, about 22.0 hours, about 22.5 hours, about 23.0 hours, about 23.50 hours, or about 24.0 hours.

In some aspects, the formulations described herein are substantially bioequivalent to D.H.E. 45®.

In some aspects, the mean Tₘₐₓ of the formulations described herein is about 80% to about 125% of the Tₘₐₓ of D.H.E. 45®.

In some aspects, the mean Tₘₐₓ of the formulations described herein is about 75% to about 130% of the Tₘₐₓ of D.H.E. 45®.

In some aspects, the mean Tₘₐₓ of the formulations described herein is about 70% to about 135% of the Tₘₐₓ of D.H.E. 45®.

In some aspects, the mean Tₘₐₓ of the formulations described herein is about 65% to about 140% of the Tₘₐₓ of D.H.E. 45®.

In some aspects, the mean Tₘₐₓ of the formulations described herein is about 60% to about 145% of the Tₘₐₓ of D.H.E. 45®.

In some aspects, the mean Tₘₐₓ of the formulations described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the Tₘₐₓ of D.H.E. 45®.

In some aspects, the mean Cₘₐₓ of the formulations described herein is about 80% to about 125% of the Cₘₐₓ of D.H.E. 45®.

In some aspects, the mean Cₘₐₓ of the formulations described herein is about 75% to about 130% of the Cₘₐₓ of D.H.E. 45®.

In some aspects, the mean Cₘₐₓ of the formulations described herein is about 70% to about 135% of the Cₘₐₓ of D.H.E. 45®.

In some aspects, the mean Cₘₐₓ of the formulations described herein is about 65% to about 140% of the Cₘₐₓ of D.H.E. 45®.

In some aspects, the mean Cₘₐₓ of the formulations described herein is about 60% to about 145% of the Cₘₐₓ of D.H.E. 45®.

In some aspects, the mean Cₘₐₓ of the formulations described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the Cₘₐₓ of D.H.E. 45®.

In some aspects, the mean AUC_{0-∞} of the formulations described herein is about 80% to about 125% of the AUC_{0-∞} of D.H.E. 45®.

In some aspects, the mean AUC_{0-∞}of the formulations described herein is about 75% to about 130% of the AUC_{0-∞} of D.H.E. 45®.

In some aspects, the mean AUC_{0-∞} of the formulations described herein is about 70% to about 135% of the AUC_{0-∞} of D.H.E. 45®.

In some aspects, the mean AUC_{0-∞} of the formulations described herein is about 65% to about 140% of the AUC_{0-∞} of D.H.E. 45®.

In some aspects, the mean AUC_{0-∞}of the formulations described herein is about 60% to about 145% of the AUC_{0-∞} of D.H.E. 45®.

In some aspects, the mean AUC_{0-∞} of the formulations described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the AUC_{0-∞} of D.H.E. 45®.The following examples are illustrative and do not limit the scope of the claimed aspects.

### EXAMPLES

### Examples 1-6: Exemplary Formulations

Table 1 provides formulation details of six exemplary DHE mesylate formulations (Examples 1-6) of the present disclosure.

TABLE 1
Exemplary DHE Mesylate Formulations

| | Example 1 | | Example 2 | | Example 3 | | Example 4 | | Example 5 | | Example 6 | |
|---|---|---|---|---|---|---|---|---|---|---|---|---|
| DHE | 3-6 | mg/mL | 3-6 | mg/mL | 3-6 | mg/mL | 3-6 | mg/mL | 3-6 | mg/mL | 3-6 | mg/mL |
| mesylate | | | | | | | | | | | | |
| caffeine | 5-15 | mg/mL | 5-15 | mg/mL | 5-15 | mg/mL | 5-15 | mg/mL | 5-15 | mg/mL | 5-15 | mg/mL |
| an isomer of | 1-3 | mg/mL | 1-3 | mg/mL | 1-3 | mg/mL | 1-3 | mg/mL | 1-3 | mg/mL | 1-3 | mg/mL |
| cresol (e.g., | | | | | | | | | | | | |
| o-cresol, m- | | | | | | | | | | | | |
| cresol, or p- | | | | | | | | | | | | |
| cresol) | | | | | | | | | | | | |
| ethanol | 3-10 | mg/mL | 3-10 | mg/mL | 3-10 | mg/mL | 3-10 | mg/mL | 3-10 | mg/mL | 3-10 | mg/mL |

| | | | | | | | |
|---|---|---|---|---|---|---|---|
| dextrose | 1-20 | mg/mL | — | — | — | — | — |

| | | | | | | | |
|---|---|---|---|---|---|---|---|
| glycerin | — | 1-20 | mg/mL | — | — | — | — |

| | | | | | | | |
|---|---|---|---|---|---|---|---|
| mannitol | — | — | 20-50 | mg/mL | — | — | — |

| | | | | | | | | | |
|---|---|---|---|---|---|---|---|---|---|
| sucrose | — | — | — | 2-20 | mg/mL | 2-20 | mg/mL | 2-20 | mg/mL |

| | | | | | | | |
|---|---|---|---|---|---|---|---|
| sodium | — | — | — | — | 0.2-4.0 | mg/mL | — |
| metabisulfite | | | | | | | |

| | | | | | | | |
|---|---|---|---|---|---|---|---|
| sodium | — | — | — | — | — | 0.1-4.0 | mg/mL |
| citrate | | | | | | | |

### Example 7: Method of Preparing the DHE Mesylate Formulations of Examples 1-6

A general procedure of preparing a DHE mesylate formulation of the present disclosure is provided as follows.

1. WFI is first placed in a suitable sterile container.

2. A chosen quantity of an isomer of cresol (e.g., o-cresol, m-cresol, or p-cresol) is added to the container with WFI and dissolved until a clear solution is obtained.

3. A chosen quantity of caffeine is added, and mixed to obtain clear solution.

4. A chosen quantity of a suitable osmotic agent is added, and mixed to obtain a clear solution. The osmotic agent can be selected from the group consisting of: dextrose, glycerin, mannitol, and sucrose, or combinations thereof.

5. Optionally, a chosen quantity of an antioxidant can be added, and mixed to obtain a clear solution. The antioxidant can be selected from the group consisting of: methionine, monothioglycerol, sodium bisulfite, sodium metabisulfite, sodium thiosulfate, sodium citrate, and thiourea, or combinations thereof.

6. An amount of methanesulfonic acid, carbon dioxide, and/or sodium hydroxide is added to reach the target pH of about 2.5-4.5.

7. An amount of a pharmaceutically acceptable alcohol (here, ethanol; optionally, propylene glycol or a polyethylene glycol) is added to the resulting solution, and mixed to obtain a homogenous solution.

8. A desired amount of DHE is added slowly with gentle mixing to obtain a clear solution.

9. An amount of methanesulfonic acid, carbon dioxide, and/or sodium hydroxide is again added to reach the target pH of about 2.5-4.5.

10. WFI is added to the resulting solution to obtain the desired final concentration of DHE.

11. The resulting solution is aseptically filtered into a pre-sterilized vessel.

12. The sterile solution is placed in a sterilized cartridge under an inert atmosphere comprising carbon dioxide and/or nitrogen.

### Example 8: Solubility and Stability Analysis

The examples herein describe analysis of formulations comprising DHE, and analysis of impurity content. Unless specifically indicated otherwise, analysis was carried out by HPLC according to the parameters in Tables 2-4.

TABLE 2
HPLC Analysis Parameters

| | |
|---|---|
| System: | Waters HPLC System equipped with a UV or |
| | PDA detector capable of detection at |
| | wavelength 280 or equivalent (for DHE), or at |
| | wavelength 220 or equivalent (for impurities). |
| HPLC Column: | Waters X-Bridge C₁₈, 250 × 4.6 mm, 5 μm Part |
| | # 186003117 or equivalent |
| Flow Rate: | 1.5 mL/min |
| Elution Mode: | Gradient |
| Detector | 280 nm (DHE) or 220 nm (impurities) |
| wavelength: | |
| Column | 40° C. |
| temperature: | |
| Sample | 25° C. |
| temperature: | |
| Injection volume: | 10 μL |
| Run Time: | 30 minutes |

TABLE 3
Gradient for HPLC Analysis of DHE

| Time | % Mobile | % Mobile | |
|---|---|---|---|
| (Minutes) | Phase A | Phase B | Curve |
| 0 | 58 | 42 | — |
| 18 | 40 | 60 | 6 |
| 25 | 58 | 42 | 6 |
| 30 | 58 | 42 | 6 |

Mobile Phase A in Tables 3 and 4 contains 2.75 g of sodium 1-heptane sulfonate in 1000 mL Purified Water, pH adjusted to 2.0 with o-phosphoric acid. Mobile Phase B in Tables 3 and 4 contains 200 mL of Mobile Phase A mixed with 800 mL of acetonitrile.

TABLE 4
Gradient for HPLC Analysis of Impurities

| Time | % Mobile | % Mobile | |
|---|---|---|---|
| (Minutes) | Phase A | Phase B | Curve |
| 0 | 58 | 42 | — |
| 18 | 40 | 60 | 6 (linear) |
| 25 | 58 | 42 | 6 (linear) |
| 30 | 58 | 42 | 6 (linear) |

Solvents that are suitable for parenteral injections were selected, and solutions of 5 mg/mL DHE mesylate in each (Table 5) were stored at 40° C. in sealed vials. Following 1 week of storage, the remaining amount of DHE was determined by HPLC, indicating solvents compatible with stable DHE storage.

TABLE 5
5 mg/mL DHE Mesylate Stability in Solvents
Suitable for Parenteral Injection

| | | | Total | |
|---|---|---|---|---|
| | DHE (mg/mL) | | Impurities | |
| | | After | | After |
| Solvent | Initial | 1 Week | Initial | 1 Week |
| Purified water | 4.1 | 4.4 | 0.24 | 3.5 |
| Alcohol, USP | 5 | 5 | 0.24 | 0.38 |
| Glycerin, USP | 1.3 | 6 | 0.21 | 0.33 |
| N-methyl pyrrolidone | 5.1 | 4.8 | 0.33 | 1.14 |
| PEG 200 | 4.3 | 4.9 | 0.25 | 1.36 |
| Propylene Glycol | 4.8 | 5.1 | 0.26 | 0.42 |
| DMSO | 5.1 | 5 | 0.26 | 0.47 |
| Glycerin:Ethanol (1:1) | 5.1 | 5.1 | 0.28 | 0.4 |
| 15% solution of Kollidon 30 | 1.7 | 3.7 | 0.24 | 9.95 |
| Benzyl Alcohol | 4.9 | 4.2 | 9.58 | 2.51 |
| N,N Dimethyl Acetamide | 5.1 | 5.4 | 0.13 | 0.8 |

### Example 9: Antioxidant and Preservative Studies

Based on the solubility and stability results of Example 8, DHE mesylate was dissolved in certain tested solvents along with antioxidants and preservatives. The formulations “A”, “D”, and “F” (Table 6) were prepared, stored at 40° C. in sealed vials, and tested for the amount of DHE and impurities after two weeks of storage (Table 3).

TABLE 6
Stability of DHE in Antioxidant and Preservative Studies

| Ingredients | | | |
|---|---|---|---|
| (weight | | | |
| by weight) | A | D | F |
| Water | 50 | 50 | 50 |
| Ethanol | 20 | — | 20 |
| Glycerin | 30 | — | 25 |
| Propylene Glycol | — | — | 5 |
| DMSO | — | 50 | — |
| Preservative | 10 mg/ml | Mixture of | 2 mg/mL |
| | benzyl alcohol | 4 mg/ml | m-cresol |
| | | methylparaben | |
| | | and 2 mg/ml | |
| | | propylparaben | |
| Anti-oxidant | 2 mg/mL | 2 mg/mL | 1.5 mg/mL |
| | monothioglycerol | citric acid | methionine |
| DHE mesylate | 5 mg/mL | 5 mg/mL | 5 mg/mL |
| pH of Finished | 4.34 | 3.44 | 4.71 |
| product before | | | |
| Adjustment | | | |
| % DHE (2 Weeks | 100.4 | 95 | 93.7 |
| old) | | | |
| % Total Impurities | 0.83 | 0.66 | 0.3 |
| after 2 weeks | | | |

### Example 10: Specific Impurity Analysis

Formulations A, D, and F of Example 9 were analyzed by HPLC for the presence of specific impurities following about 10-14 days of storage in sealed vials at 40° C. (Table 7).

TABLE 7

| Retention | | | | |
|---|---|---|---|---|
| Time | Relative | | | |
| (RT) | RT | A | D | F |
| 2.46 | 0.43 | | | |
| 2.66 | 0.46 | | | |
| 2.84 | 0.49 | | | |
| 3.15 | 0.54 | 0.03 | | |
| 3.45 | 0.6 | 0.16 | | 0.1 |
| 3.92 | 0.68 | 0.06 | 0.3 | 0.1 |
| | (Imp-D) | | | |
| 4.23 | 0.75 | 0.33 | 0.1 | |
| 4.86 | 0.84 | 0.1 | 0 | |
| | (Imp-C) | | | |
| 5.2 | 0.9 | 0.05 | 0.1 | |
| 6.59 | 1.14 | | | |
| 7.16 | 1.24 | 0.09 | 0.1 | 0.1 |
| | (Imp-B) | | | |
| 8.71 | 1.50 | | | |
| | (Imp-E) | | | |

| | | | |
|---|---|---|---|
| % Total Impurities | 0.82 | 0.6 | 0.3 |
| (10-14 days) | | | |
| % DHE (10-14 days) | 100 | 95 | 94 |

### Example 11: Formula J

An additional formulation “J” was prepared according to the components and amounts listed in Table 8. After one day of storage at room temperature, formulation J was analyzed for DHE and impurities, as shown in the last two rows of Table 8. Testing for specific impurities relative to a sample of API (DHE mesylate) yielded the results of Table 9.

TABLE 8
Formulation J Components and HPLC Analysis

| Ingredients | |
|---|---|
| (weight by weight) | J |
| Water | 50 |
| Ethanol | 20 |
| Glycerin | 25 |
| Propylene Glycol | 5 |
| DMSO | — |
| Preservative | 10 mg/mL Benzyl Alcohol |
| Antioxidant | Monothioglycerol 2 mg/mL |
| DHE Mesylate | 5 mg/mL |
| pH of Finished product | 4.42 |
| before Adjustment | |
| pH of Finished product | 3.59 |
| After Adjustment (0.1% | |
| Methanesulfonic Acid/ | |
| 0.05N NaOH Solution) | |
| % DHE (1 day following | 100.4 |
| storage) | |
| % Total Impurities (1 | 0.18 |
| day following storage) | |

TABLE 8
HPLC Analysis of Formulation J

| RRT | API | J |
|---|---|---|
| 0.43 | | |
| 0.46 | | |
| 0.49 | | |
| 0.54 | | |
| 0.60 | | 0.04 |
| 0.68 (Imp-D) | | |
| 0.75 | 0.03 | 0.04 |
| 0.84 (Imp-C) | 0.02 | 0.04 |
| 0.9 | 0.03 | |
| 0.91 | | |
| 1.14 | | |
| 1.24 | 0.11 | 0.1 |
| Total Impurities following | 0.19 | 0.18 |
| 1 day of storage | | |

Values less than 0.05 are not significant. Because the total number of impurities was unchanged following storage, indicating the starting and final amount of impurities is the same, this demonstrates the formulation is stable.

### Example 12: Further Preservative and Antioxidant Studies

Additional formulations “L”, “M”, and “N” were prepared according to Table 9 and analyzed for the concentration of DHE and impurities following one day in sealed vials at 40° C. Specific impurities were analyzed after storage in sealed vials after one and two weeks at 40° C. (Table 10).

TABLE 9
Further Preservative and Antioxidant Formulations

| Ingredients | | | |
|---|---|---|---|
| (weight by | | | |
| weight) | L | M | N |
| Water | 50 | 50 | 50 |
| Ethanol | 20 | 20 | 20 |
| Glycerin | 25 | 25 | 25 |
| Propylene Glycol | 5 | 5 | 5 |
| DMSO | — | — | — |
| Preservative | 2 mg/mL | — | 10 mg/ml |
| | m-cresol | | benzyl alcohol |
| Antioxidant | — | 1.5 mg/mL | — |
| | | methionine | |
| DHE mesylate | 4 mg/mL | 4 mg/mL | 4 mg/mL |
| API Dissolved | Yes | Yes | Yes |
| Completely | | | |
| Visually | | | |
| pH of Finished | 3.59 | 3.6 | 3.6 |
| product After | | | |
| Adjustment (1% | | | |
| Methanesulfonic | | | |
| Acid) | | | |
| % DHE (1 day, | 95.3 | 95.7 | 97.4 |
| 40° C.) | | | |
| % DHE (1 week, | 97.7 | 97 | 99 |
| 40° C.) | | | |
| % Total | 0.32 | 0.51 | 0.74 |
| Impurities (1 | | | |
| week, 40° C.) | | | |

TABLE 10
Stability Analysis of Formulations L, M, and N Following Storage
at Accelerated Conditions (ACC) (40° C., sealed vials)

| Relative | | | | | | | | | | |
|---|---|---|---|---|---|---|---|---|---|---|
| Retention | | | | | | | | | | |
| Time | | Initial | | | 1 Week ACC | | | 2 Weeks ACC | | |
| (RRT) | API | L | M | N | L | M | N | L | M | N |
| 0.36 | | | | | 0.02 | 0.02 | | | 0.02 | 0.02 |
| 0.4 | | | | | 0.03 | | | 0.02 | | |
| 0.43 | | 0.02 | | | | | | | | |
| 0.54 | | | | | | | | | | |
| 0.56 | | | | | | | | | | 0.06 |
| 0.59 | | | | | | | | | | |
| 0.61 | | 0.01 | | | | | | | | |
| 0.68 | | | | | 0.02 | 0.04 | 0.23 | 0.04 | 0.08 | 0.32 |
| 0.74 | | 0.02 | 0.02 | 0.02 | 0.16 | 0.3 | 0.24 | 0.29 | 0.49 | 0.39 |
| (Impurity D) | | | | | | | | | | |
| 0.77 | | 0.01 | 0.02 | 0.02 | | | 0.12 | | 0.04 | |
| 0.8 | | | | | | | | | | 0.33 |
| 0.87 | 0 | | 0.04 | 0.03 | 0.04 | 0.05 | 0.03 | | 0.04 | 0.04 |
| 0.92 | 0.1 | 0.03 | 0.05 | 0.04 | 0.02 | 0.03 | 0.04 | | 0.04 | 0.04 |
| 1.22 | 0.1 | 0.07 | 0.09 | 0.07 | 0.08 | 0.09 | 0.08 | 0.1 | 0.08 | 0.07 |
| (Impurity B) | | | | | | | | | | |
| 1.49 | | | | | | | | | | 0.04 |
| % Total | 0.2 | 0.16 | 0.22 | 0.18 | 0.32 | 0.51 | 0.74 | 0.43 | 0.77 | 1.25 |
| Impurities | | | | | | | | | | |
| % DHE | N/A | 95.3 | 95.74 | 97.43 | 97.7 | 97 | 99 | 95.63 | 95.31 | 95.14 |

Specification limits: not more than 1.0% (specified impurity, e.g., Impurity D), not more than 0.5% (unspecified impurity), and not more than 4.0% (total impurities).

Impurities were within specification limits, indicating stability.

### Example 13: Formulation for Animal Study

The formulation of Table 11 (“P”) was prepared for use in canine studies. An additional formulation was prepared (“O”), replacing DMSO of formula P with an equal amount of propylene glycol. Specific impurity analysis of formulations O and P are shown in Table 12.

TABLE 11
Formulation P for Canine Studies

| Ingredients (% w/w) | P |
|---|---|
| Water | 50 |
| Ethanol | 20 |
| Glycerin | 25 |
| DMSO | 5 |
| Preservative | 1.5 mg/mL m-cresol |
| Antioxidant | 1.5 mg/mL methionine |
| DHE mesylate | 4 mg/mL |
| API Dissolved | Yes |
| Completely Visually | |
| pH of Finished product | 3.62 |
| After Adjustment (1% | |
| Methanesulfonic Acid) | |
| % DHE | 100 |
| % Total Impurities | 0.12 |

TABLE 12
Specific Impurity Analysis of Formulations O and P

| | | 0 Time | | 2 Weeks 40° C. | | 1 Month 40° C. | | 1 Month 30° C. | | 1 Month 25° C. | |
|---|---|---|---|---|---|---|---|---|---|---|---|
| RRT | API | O | P | O | P | O | P | O | P | O | P |
| 0.39 | | | | 0.02 | 0.02 | 0.05 | 0.04 | | | | |
| 0.43 | | | | | 0.02 | | | | | | |
| 0.58 | | | | | | 0.04 | 0.07 | | | | |
| 0.67 | | | | 0.03 | 0.05 | 0.08 | 0.15 | 0.04 | | | |
| 0.74 | | 0.02 | 0 | 0.59 | 0.66 | 1.14 | 1.33 | 0.3 | 0.35 | 0.1 | 0.12 |
| (Imp-D) | | | | | | | | | | | |
| 0.77 | | 0.01 | 0 | | | | | | | | |
| 0.87 | 0 | | | | 0.02 | | 0.03 | 0.03 | 0.03 | | |
| 0.92 | 0.1 | 0.03 | 0 | | 0.02 | 0.04 | 0.06 | 0.03 | | 0.04 | 0.03 |
| 1.21 | 0.1 | 0.07 | 0.1 | 0.11 | 0.07 | 0.1 | 0.1 | 0.08 | 0.1 | 0.1 | 0.11 |
| (Imp-B) | | | | | | | | | | | |
| 1.49 | | | | 0.02 | | | | | | | |
| (Imp-E) | | | | | | | | | | | |
| % Total | 0.2 | 0.13 | 0.1 | 0.77 | 0.86 | 1.45 | 1.78 | 0.48 | 0.48 | 0.24 | 0.26 |
| Impurities | | | | | | | | | | | |
| % DHE | N/A | 100 | 100 | 99.9 | 99.7 | 98.2 | 96.9 | 99.5 | 98.6 | 99.6 | 98.3 |

### Example 14: Animal Study

Formulation P was injected intravenously, and pharmacokinetic data were generated (Table 14). The United States Food and Drug Administration approved reference product of Table 13 (manufactured by PERRIGO COMPANY PLC®) was used as a comparator.

TABLE 13
Reference Product for Dog Study

| Ingredients (% w/w) | |
|---|---|
| Water | 50 |
| Ethanol | 20 |
| Glycerin | 25 |
| Propylene Glycol | — |
| DMSO | 5 |
| Preservative | 1.5 mg/mL m-cresol |
| Antioxidant | 1.5 mg/mL methionine |
| DHE Mesylate | 4 mg/mL |
| pH of Finished product | 4.58 |
| before Adjustment | |
| pH of Finished product | 3.62 |
| After Adjustment (1% | |
| Methanesulfonic Acid) | |
| % DHE | 100 |
| % Total Impurities | 0.12 |

TABLE 14
Time Dependent Mean Plasma Concentration
of Dog Formulation and Reference Product

Mean Plasma Concentration (ng/ml)

| | Reference Product | |
|---|---|---|
| Time | (Table 13) | Formula P |
| (hours) | N = 1 | N = 3 |
| 0 | 0 | 0 |
| 0.08 | 46.79 | 45.03 |
| 0.25 | 21.76 | 18.85 |
| 0.5 | 15.84 | 13.6 |
| 0.75 | 11.78 | 10.27 |
| 1 | 9.05 | 9.09 |
| 1.5 | 5.33 | 5.7 |
| 2 | 5.1 | 4.86 |
| 2.5 | 4.01 | 3.64 |
| 3 | 2.99 | 2.64 |
| 4 | 2.42 | 2.32 |
| 5 | 1.69 | 1.19 |
| 6 | 1.14 | 0.79 |
| 8 | 0 | 0.42 |

These data show that formulation P exhibits a similar mean plasma concentration over time in dogs when compared to the reference product of Table 13 manufactured by PERRIGO COMPANY PLC®.

### Examples 15-29: Additional Stable Formulations

It was observed that DHE is highly soluble in DMSO, benzyl alcohol, ethanol, propylene glycol, and captisol. Based on this discovery and the solubility and stability data generated above for various solvents, preservatives, and antioxidants, additional stable formulations are included as non-limiting examples in Table 15. Prophetic formulations are included as non-limiting examples in Tables 16 and 17.

TABLE 15
Stable DHE Formulations

| Ingredient | Example | Example | Example | Example | Example |
|---|---|---|---|---|---|
| (mg) | 15 | 16 | 17 | 18 | 19 |
| DHE | 4 | 4 | 4 | 4 | 4 |
| Metacresol | 1.5 | 1.5 | 1.5 | 1.5 | 1.5 |
| Methionine | 1.5 | 1.5 | 1.5 | 1.5 | 1.5 |
| Dimethyl | 540 | 200 | — | 540 | 540 |
| Sulfoxide | | | | | |
| Glycerin | 190 | — | — | — | — |
| Ethanol | — | — | — | 190 | 190 |
| Dextrose | — | — | — | — | 50 |
| Captisol | — | 150 | 150 | — | — |
| Water | 1 mL | 1 mL | 1 mL | 1 mL | 1 mL |
| (quantity | | | | | |
| sufficient) | | | | | |
| Total | 0.26% | 0.30% | 0.33% | 0.22% | 0.22% |
| impurities | | | | | |

TABLE 16
Prophetic DHE Formulations

| Ingredient | Example | Example | Example | Example | Example |
|---|---|---|---|---|---|
| (mg) | 20 | 21 | 22 | 23 | 24 |
| DHE | 4 | 4 | 4 | 4 | 4 |
| Metacresol | 1.5 | 1.5 | 1.5 | 1.5 | 1.5 |
| Methionine | 1.5 | 1.5 | 1.5 | 1.5 | 1.5 |
| Dimethyl | 750 | — | — | 750 | 540 |
| Sulfoxide | | | | | |
| Glycerin | — | — | — | — | — |
| Propylene | 100 | | — | | |
| Glycol | | | | | |
| Ethanol | — | — | — | 100 | 250 |
| Dextrose | — | — | 50 | 50 | 50 |
| Captisol | — | 500 | 150 | — | — |
| Trehalose | 50 | | | | |
| Water | 1 mL | 1 mL | 1 mL | 1 mL | 1 mL |
| (quantity | | | | | |
| sufficient) | | | | | |

TABLE 17
Prophetic DHE Formulations

| Ingredient | Example | Example | Example | Example | Example |
|---|---|---|---|---|---|
| (mg) | 25 | 26 | 27 | 28 | 29 |
| DHE | 4 | 4 | 4 | 4 | 4 |
| Metacresol | 1.5 | 1.5 | 1.5 | 1.5 | 1.5 |
| Methionine | 1.5 | 1.5 | 1.5 | 1.5 | 1.5 |
| Dimethyl | 750 | — | — | 750 | 540 |
| Sulfoxide | | | | | |
| Glycerin | — | — | — | — | — |
| Ethanol | — | — | — | 100 | 250 |
| Captisol | — | 500 | 150 | — | — |
| Trehalose | 25 | | 50 | | |
| Caffeine | 15 | 15 | | 15 | |
| Water | 1 mL | 1 mL | 1 mL | 1 mL | 1 mL |
| (quantity | | | | | |
| sufficient) | | | | | |

What is claimed is:

1. A pre-filled injector comprising:
a pharmaceutically acceptable formulation comprising:
dihydroergotamine mesylate at a concentration of about 3 mg/mL to about 6 mg/mL; and
carbon dioxide at a concentration sufficient to retard oxidative degradation of the dihydroergotamine mesylate.

2. The pre-filled injector of claim 1, wherein the pharmaceutically acceptable formulation further comprises caffeine.

3. The pre-filled injector of claim 2, wherein the concentration of caffeine is about 5 mg/mL to about 15 mg/mL.

4. The pre-filled injector of any one of claims 1-3, wherein the pharmaceutically acceptable formulation further comprises an osmotic agent.

5. The pre-filled injector of claim 4, where the osmotic agent is selected from the group consisting of: dextrose, glycerin, mannitol, and sucrose, or combinations thereof.

6. The pre-filled injector of claim 5, where the osmotic agent is dextrose at a concentration of about 1 mg/mL to about 20 mg/mL.

7. The pre-filled injector of claim 5, where the osmotic agent is glycerin at a concentration of about 1 mg/mL to about 20 mg/mL.

8. The pre-filled injector of claim 5, where the osmotic agent is mannitol at a concentration of about 20 mg/mL to about 50 mg/mL.

9. The pre-filled injector of claim 5, where the osmotic agent is sucrose at a concentration of about 2 mg/mL to about 20 mg/mL.

10. The pre-filled injector of any one of claims 1-9, where the volume of the pharmaceutically acceptable formulation to be dispensed from the pre-filled injector is adjustable to about 0.1 mL to about 0.3 mL per injection.

11. The pre-filled injector of any one of claims 1-10, wherein the pharmaceutically acceptable formulation further comprises an antioxidant.

12. The pre-filled injector of claim 11, wherein the antioxidant is selected from the group consisting of: methionine, monothioglycerol, sodium bisulfite, sodium metabisulfite, sodium thiosulfate, sodium citrate, and thiourea, or combinations thereof.

13. The pre-filled injector of claim 12, wherein the antioxidant is methionine at a concentration of about 1.5 mg/mL to about 5 mg/mL.

14. The pre-filled injector of claim 13, wherein the antioxidant is methionine at a concentration of about 1.5 mg/mL.

15. The pre-filled injector of claim 12, wherein the antioxidant is monothioglycerol.

16. The pre-filled injector of claim 15, wherein the antioxidant is monothioglycerol at a concentration of about 2 mg/mL to about 3 mg/mL.

17. The pre-filled injector of claim 15 or claim 16, wherein the antioxidant is monothioglycerol at a concentration of about 2 mg/mL.

18. The pre-filled injector of claim 12, wherein the antioxidant is sodium metabisulfite at a concentration of about 0.2 mg/mL to about 4.0 mg/mL.

19. The pre-filled injector of claim 12, wherein the antioxidant is sodium citrate at a concentration of about 0.1 mg/mL to about 4.0 mg/mL.

20. The pre-filled injector of any one of claims 1-19, wherein the pharmaceutically acceptable formulation is contained within an about 3.0 mL sterilized cartridge.

21. The pre-filled injector of any one of the claims 1-20, wherein the pharmaceutically acceptable formulation has a pH of about 2.5 to about 4.5.

22. The pre-filled injector of any one of claims 1-21, wherein the pharmaceutically acceptable formulation further comprises a preservative.

23. The pre-filled injector of claim 22, wherein the preservative is an isomer of cresol selected from the group consisting of o-cresol, m-cresol, and p-cresol, or combinations thereof.

24. The pre-filled injector of claim 23, wherein the preservative is o-cresol.

25. The pre-filled injector of claim 23, wherein the preservative is m-cresol.

26. The pre-filled injector of claim 25, wherein m-cresol is present at a concentration of about 1 mg/mL to about 5 mg/mL.

27. The pre-filled injector of claim 25 or claim 26, wherein m-cresol is present at a concentration of about 1 mg/mL to about 2.5 mg/mL.

28. The pre-filled injector of any one of claims 25-27, wherein m-cresol is present at a concentration of about 1.5 mg/mL.

29. The pre-filled injector of claim 23, wherein the preservative is p-cresol.

30. The pre-filled injector of claim 22, wherein the preservative is benzyl alcohol.

31. The pre-filled injector of claim 30, wherein benzyl alcohol is present at a concentration of about 5 mg/mL to about 100 mg/mL.

32. The pre-filled injector of claim 31, wherein benzyl alcohol is present at a concentration of about 10 mg/mL.

33. The pre-filled injector of claim 22, wherein the preservative is a composition of one or more parabens.

34. The pre-filled injector of claim 33, wherein the composition of one or more parabens comprises methylparaben.

35. The pre-filled injector of claim 33 or claim 34, wherein the composition of one or more parabens comprises propylparaben.

36. The pre-filled injector of any one of claims 33-35, wherein the composition of one or more parabens comprises methylparaben and propylparaben.

37. The pre-filled injector of any one of claims 1-36, wherein the pharmaceutically acceptable formulation is stable.

38. A pharmaceutically acceptable formulation comprising:
about 3 mg/mL to about 6 mg/mL dihydroergotamine mesylate;
about 3 mg/mL to about 10 mg/mL of a pharmaceutically acceptable alcohol;
about 5 mg/mL to about 15 mg/mL caffeine;
about 1 mg/mL to about 3 mg/mL an isomer of cresol selected from the group consisting of o-cresol, m-cresol, and p-cresol, or combinations thereof;
carbon dioxide at a concentration sufficient to retard oxidative degradation of the dihydroergotamine mesylate; and
an osmotic agent selected from the group consisting of:
dextrose, glycerin, mannitol, and sucrose , or combinations thereof.

39. The pharmaceutically acceptable formulation of claim 38, where the pharmaceutically acceptable alcohol is selected from the group consisting of: propylene glycol, ethanol, and a pharmaceutically acceptable polyethylene glycol, or combinations thereof.

40. The pharmaceutically acceptable formulation of claim 39, where the pharmaceutically acceptable alcohol is ethanol.

41. The pharmaceutically acceptable formulation of any one of claims 38-40, where the isomer of cresol is o-cresol.

42. The pharmaceutically acceptable formulation of any one of claims 38-40, where the isomer of cresol is m-cresol.

43. The pharmaceutically acceptable formulation of any one of claims 38-40, where the isomer of cresol is p-cresol.

44. The pharmaceutically acceptable formulation of any one of claims 38-43, where the osmotic agent is dextrose at a concentration of about 1 mg/mL to about 20 mg/mL.

45. The pharmaceutically acceptable formulation of any one of claims 38-43, where the osmotic agent is glycerin at a concentration of about 1 mg/mL to about 20 mg/mL.

46. The pharmaceutically acceptable formulation of any one of claims 38-43, where the osmotic agent is mannitol at a concentration of about 20 mg/mL to about 50 mg/mL.

47. The pharmaceutically acceptable formulation of any one of claims 38-43, where the osmotic agent is sucrose at a concentration of about 2 mg/mL to about 20 mg/mL.

48. The pharmaceutically acceptable formulation of any one of claims 38-47, wherein the pharmaceutically acceptable formulation further comprises an antioxidant.

49. The pharmaceutically acceptable formulation of claim 48, wherein the antioxidant is selected from the group consisting of: methionine, monothioglycerol, sodium bisulfate, sodium metabisulfite, sodium thiosulfate, sodium citrate, and thiourea, or combinations thereof.

50. The pharmaceutically acceptable formulation of claim 49, where in the antioxidant is methionine at a concentration of about 1.5 mg/mL to about 5 mg/mL.

51. The pharmaceutically acceptable formulation of claim 49 or claim 50, where in the antioxidant is methionine at a concentration of about 1.5 mg/mL.

52. The pharmaceutically acceptable formulation of claim 49, where in the antioxidant is monothioglycerol at a concentration of about 2 mg/mL to about 3 mg/mL.

53. The pharmaceutically acceptable formulation of claim 52, where in the antioxidant is monothioglycerol at a concentration of about 2 mg/mL.

54. The pharmaceutically acceptable formulation of claim 49, where in the antioxidant is sodium metabisulfite at a concentration of about 0.2 mg/mL to about 4.0 mg/mL

55. The pharmaceutically acceptable formulation of claim 49, where in the antioxidant is sodium citrate at a concentration of about 0.1 mg/mL to 4.0 mg/mL

56. The pharmaceutically acceptable formulation of any one of claims 38-55, wherein the pharmaceutically acceptable formulation further comprises a cyclodextrin.

57. The pharmaceutically acceptable formulation of claim 56, wherein the cyclodextrin is selected from the group consisting of: 2-hydroxypropyl-β-cyclodextrin, O-methyl-β-cyclodextrin, and γ-cyclodextrin, or combinations thereof.

58. The pharmaceutically acceptable formulation of any one of claims 38-57, wherein the pharmaceutically acceptable formulation is stable.

59. A pre-filled injector comprising the pharmaceutically acceptable formulation of any one of claims 38-58.

60. The pre-filled injector of claim 59, wherein the volume of the formulation to be dispensed from the pre-filled injector is adjustable to about 0.1 mL to about 0.3 mL per injection.

61. The pre-filled injector of claim 59 or claim 60, wherein the pharmaceutically acceptable formulation is contained within an about 3.0 mL sterilized cartridge.

62. A method of treating a migraine or a cluster headache attack in a patient, comprising:
parenterally administering to the patient in need thereof a therapeutically effective amount of a pharmaceutically acceptable formulation comprising:
dihydroergotamine mesylate at a concentration of about 3 mg/mL to about 6 mg/mL; and
carbon dioxide at a concentration sufficient to retard oxidative degradation of the dihydroergotamine mesylate.

63. The method of claim 62, wherein the pharmaceutically acceptable formulation further comprises caffeine.

64. The method of claim 63, where the concentration of caffeine in the pharmaceutically acceptable formulation is about 5 mg/mL to about 15 mg/mL.

65. The method of any one of claims 62-64, wherein the pharmaceutically acceptable formulation further comprises an osmotic agent selected from the group consisting of: dextrose, glycerin, mannitol, and sucrose, or combinations thereof.

66. The method of claim 65, where the osmotic agent is dextrose at a concentration of about 1 mg/mL to about 20 mg/mL.

67. The method of claim 65, where the osmotic agent is glycerin at a concentration of about 1 mg/mL to about 20 mg/mL.

68. The method of claim 65, where the osmotic agent is mannitol at a concentration of about 20 mg/mL to about 50 mg/mL.

69. The method of claim 65, where the osmotic agent is sucrose at a concentration of about 2 mg/mL to about 20 mg/mL.

70. The method of any one of claims 62-69, wherein the pharmaceutically acceptable formulation further comprises an antioxidant.

71. The method of claim 70, where the antioxidant is selected from the group consisting of: methionine, monothioglycerol, sodium bisulfite, sodium metabisulfite, sodium thiosulfate, sodium citrate, and thiourea, or combinations thereof.

72. The method of claim 71, where the antioxidant is methionine at a concentration of about 1.5 mg/mL to about 5 mg/mL.

73. The method of claim 72, where the antioxidant is methionine at a concentration of about 1.5 mg/mL.

74. The method of claim 71, where the antioxidant is monothioglycerol at a concentration of about 2 mg/mL to about 3 mg/mL.

75. The method of claim 74, where the antioxidant is monothioglycerol at a concentration of about 2 mg/mL.

76. The method of claim 71, where the antioxidant is sodium metabisulfite at a concentration of about 0.2 mg/mL to about 4.0 mg/mL.

77. The method of claim 71, where the antioxidant is sodium citrate at a concentration of about 0.1 mg/mL to about 4.0 mg/mL.

78. The method of any one of claims 62-77, wherein the pharmaceutically acceptable formulation further comprises a cyclodextrin.

79. The method of claim 78, where the cyclodextrin is selected from the group consisting of: 2-hydroxypropyl-β-cyclodextrin, O-methyl-β-cyclodextrin, and γ-cyclodextrin, or combinations thereof.

80. The method of any one of claims 62-79, wherein the pharmaceutically acceptable formulation further comprises a preservative.

81. The method of claim 80, wherein the preservative is an isomer of cresol selected from the group consisting of o-cresol, m-cresol, and p-cresol, or combinations thereof.

82. The method of claim 81, wherein the preservative is o-cresol.

83. The method of claim 81, wherein the preservative is m-cresol.

84. The method of claim 83, wherein m-cresol is present at a concentration of about 1 mg/mL to about 5 mg/mL.

85. The method of claim 84, wherein m-cresol is present at a concentration of about 1 mg/mL to about 2.5 mg/mL.

86. The method of claim 85, wherein m-cresol is present at a concentration of about 1.5 mg/mL.

87. The method of claim 81, wherein the preservative is p-cresol.

88. The method of claim 80, wherein the preservative is benzyl alcohol.

89. The method of claim 88, wherein benzyl alcohol is present at a concentration of about 5 mg/mL to about 100 mg/mL.

90. The method of claim 89, wherein benzyl alcohol is present at a concentration of about 10 mg/mL.

91. The method of any one of claims 62-90, wherein the pharmaceutically acceptable formulation is administered into the patient subcutaneously, intramuscularly, or intravenously.

92. The method of any one of claims 62-91, wherein the pharmaceutically acceptable formulation is administered to the patient from an injector pre-loaded with the pharmaceutically acceptable formulation.

93. The method of claim 91 or claim 92, wherein the volume of the pharmaceutically acceptable formulation to be dispensed to the patient is about 0.1 mL to about 0.3 mL.

94. The method of any one of claims 62-93, wherein the treatment reduces the severity, duration, or occurrence of headaches or migraines experienced by the patient.

95. The method of any one of claims 62-94, wherein the pharmaceutically acceptable formulation is stable and ready-to-use.

96. A method of manufacturing a pre-filled injector comprising:
preparing a pharmaceutically acceptable formulation comprising:
about 3 mg/mL to about 6 mg/mL dihydroergotamine mesylate;
about 3 mg/mL to about 10 mg/mL of a pharmaceutically acceptable alcohol;
about 5 mg/mL to about 15 mg/mL caffeine;
about 1 mg/mL to about 3 mg/mL an isomer of cresol selected from the group consisting of o-cresol, m-cresol, and p-cresol, or combinations thereof and
an osmotic agent selected from the group consisting of:
dextrose, glycerin, mannitol, and sucrose, or combinations thereof;
loading a sterile cartridge with the pharmaceutically acceptable formulation under a headspace of carbon dioxide at a concentration sufficient to retard oxidation of dihydroergotamine mesylate; and
attaching the sterile cartridge operably to an injector.

97. The method of claim 96, where the pharmaceutically acceptable alcohol is selected from the group consisting of: propylene glycol, ethanol, and a pharmaceutically acceptable polyethylene glycol, or combinations thereof.

98. The method of claim 97, where the pharmaceutically acceptable alcohol is ethanol.

99. The method of any one of claims 96-98, where the sterile cartridge has a capacity of about 3.0 mL.

100. The method of any one of claims 96-99, where the isomer of cresol is o-cresol.

101. The method of any one of claims 96-99, where the isomer of cresol is m-cresol.

102. The method of any one of claims 96-99, where the isomer of cresol is p-cresol.

103. The method of any one of claims 96-102, where the osmotic agent is dextrose at a concentration of about 1 mg/mL to about 20 mg/mL.

104. The method of any one of claims 96-102, where the osmotic agent is glycerin at a concentration of about 1 mg/mL to about 20 mg/mL.

105. The method of any one of claims 96-102, where the osmotic agent is mannitol at a concentration of about 20 mg/mL to about 50 mg/mL.

106. The method of any one of claims 96-102, where the osmotic agent is sucrose at a concentration of about 2 mg/mL to about 20 mg/mL.

107. The method of any one of claims 96-106, wherein the formulation further comprises a cyclodextrin.

108. The method of claim 107, where the cyclodextrin is selected from the group consisting of: 2-hydroxypropyl-β-cyclodextrin, O-methyl-β-cyclodextrin, and γ-cyclodextrin, or combinations thereof.

109. The method of any one of claims 96-108, where the formulation further comprises an antioxidant.

110. The method of claim 109, where the antioxidant is selected from the group consisting of:
methionine, monothioglycerol, sodium bisulfite, sodium metabisulfite, sodium thiosulfate, sodium citrate, and thiourea, or combinations thereof.

111. The method of claim 110, where the antioxidant is methionine at a concentration of about 1.5 mg/mL to about 5 mg/mL.

112. The method of claim 110, where the antioxidant is monothioglycerol at a concentration of about 2 mg/mL to about 3 mg/mL.

113. The method of claim 110, where the antioxidant is sodium metabisulfite at a concentration of about 0.2 mg/mL to about 4.0 mg/mL.

114. The method of claim 110, where the antioxidant is sodium citrate at a concentration of about 0.1 mg/mL to about 4.0 mg/mL.

115. The method of any one of claims 96-114, where the volume of the formulation to be dispensed from the pre-filled injector is adjustable to about 0.1 mL to about 0.3 mL per injection.

116. The method of any one of claims 96-116, wherein the pharmaceutically acceptable formulation is stable and ready-to-use.
